(19) United States
(12) Patent Application Publication
(10) Pub. No.: US 2015/0216794 A1
(43) Pub. Date: Aug. 6, 2015

(54) METHODS OF TREATING PAIN ASSOCIATED WITH OSTEOARTHRITIS OF A JOINT WITH A TOPICAL FORMULATION OF A SPIRO-OXINDOLE COMPOUND

(71) Applicant: IVAX International GmbH, Rapperswil (CH)

(72) Inventors: Michael Fetell, Miami Beach, FL (US); Richard Malamut, Rose Valley, PA (US); Michael J. Lamson, Cary, NC (US); Ofer Spiegelstein, Ramat Hasharon (IL); Charles Jay Cohen, Vancouver (CA); Yigal Paul Goldberg, Vancouver (CA); Rostam Namdari, Burnaby (CA); Nicola Anne Price, Qualicum Beach (CA); Katie Jane Webster Proctor, Abingdon (UK)

(21) Appl. No.: 14/614,895

(22) Filed: Feb. 5, 2015

Related U.S. Application Data
(60) Provisional application No. 61/936,221, filed on Feb. 5, 2014.
(60) Provisional application No. 62/026,554, filed on Jul. 18, 2014.

(51) Int. Cl. A61K 9/00; A61K 31/167; A61K 31/407

(52) U.S. Cl. CPC A61K 9/0014; A61K 31/407; A61K 31/167

(57) ABSTRACT

Methods of treating pain associated with osteoarthritis of a joint in a mammal with a pharmaceutical composition comprising a spiro-oxindole compound of the formula:

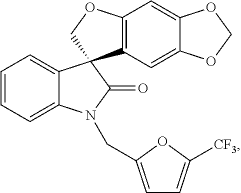 [SMILES: O=C1N(Cc2ccc(C(F)(F)F)o2)c2ccccc2[C@]12COc1cc3c(cc12)OCO3]

are disclosed. The methods provide excellent penetration of the spiro-oxindole compound into the synovial membrane of the affected joint to effectively reduce the severity of the pain and/or to alleviate the pain with minimal or negligible systemic exposure.

### FIELD OF THE INVENTION

The present invention is directed to methods of treating pain associated with osteoarthritis of a joint in a mammal, preferably a human, wherein the methods comprise administering, preferably periodically administering, to the affected joint of the mammal a topical pharmaceutical composition comprising one or more excipients and a therapeutically effective amount of a spiro-oxindole compound.

### BACKGROUND OF THE INVENTION

Osteoarthritis is the most common form of arthritis, affecting over 20 million people in the United States. Surveys in United Kingdom and worldwide reveal that 25% of persons 55 years or older have experienced knee pain on most days per month over the prior year, and half of these individuals have radiographic osteoarthritis sufficient to make the diagnosis of symptomatic osteoarthritis of the knee (Felson D T, “Clinical practice: osteoarthritis of the knee”, N Engl J Med 2006; 354:841-8; Peat G, McCarney R, Croft P, “Knee pain and osteoarthritis in older adults: a review of community burden and current use of primary health care”, Ann Rheum Dis 2001; 60:91-7). Radiologic findings of arthritis of the knee are age related, and are seen in up to one-third of older adults. The diagnosis of osteoarthritis should be based upon both clinical and radiographic criteria as 50% of those with radiographic changes of osteoarthritis do not suffer pain from the osteoarthritis, and 50% of people above age 55 years who complain of knee pain lack radiographic changes of osteoarthritis. Osteoarthritis is more common in women than men, increases with age, and is a leading cause of impaired mobility in elderly people. The prevalence of painful disabling knee osteoarthritis in people over age 55 years is approximately 10% (Peat G, op.cit.). Risk factors include obesity, prior knee injury or surgery, and occupational factors such as bending and lifting. The pain experienced by patients with osteoarthritis, i.e., pain associated with osteoarthritis of a joint, is typically related to activity and is believed to emanate from bone, synovial inflammation, or a stretched joint capsule. Common symptoms of knee osteoarthritis include pain on climbing stairs, arising from a chair, or walking long distances. Often patients complain of morning stiffness lasting for about 30 minutes. On examination, there may be tenderness at the junction of the femur and tibia, joint malalignment (varus or valgus deformity), and antalgia (limp due to avoidance of pain) (Felson D T, op.cit.).

Current recommendations for symptomatic osteoarthritis are to use acetaminophen as a first-line analgesic in reducing or alleviating the pain. However, only one-third of patients found the drug effective; whereas, over 60% responded better to the widely-used oral non-steroidal anti-inflammatory drugs (NSAIDs) (Wolfe F, Zhao S, Lane N, “Preference for nonsteroidal antiinflammatory drugs over acetaminophen by rheumatic disease patients: a survey of 1,799 patients with osteoarthritis, rheumatoid arthritis, and fibromyalgia”, Arthritis Rheum 2000; 43:378-85). Long-term use of oral NSAIDs is discouraged, however, because of, for example, gastrointestinal side effects (such as gastrointestinal ulceration and bleeding) and cardiovascular side effects due to systemic exposure of the NSAID. In addition, the combination of exercise and modest weight loss has been shown to be beneficial in a randomized controlled study (Messier S P, Loeser R F, Miller G D, Morgan T M, Rejeski W J, Sevick M A, Ettinger W H, Pahor M, Williamson J D, “Exercise and dietary weight loss in overweight and obese older adults with knee osteoarthritis: the arthritis, diet, and activity promotion trial,” Arthritis Rheum 2004; 50:1501-10).

Voltaren® (diclofenac sodium gel) is a NSAID in a topical formulation. It is a marketed treatment option for osteoarthritis patients. While the risk of gastrointestinal side effects for NSAID topical use is lower than it is for NSAID oral use, these serious side effects remain a concern for topical diclofenac. Furthermore, meaningful elevation of hepatic enzymes has recently been reported in some patients on long-term topical diclofenac, necessitating regular monitoring for hepatotoxicity in this patient population (see, FDA website, MedWatch, 2009; Volteren Gel (diclofenac sodium) 1% topical gel; Safety Labeling Changes Approved by FDA Center for Drug Evaluation and Research—September 2009).

There exists, therefore, a need for a topical treatment of pain associated with osteoarthritis of a joint which provides effective localized reduction of the severity of the pain or alleviation of the pain with minimal or negligible systemic exposure.

### SUMMARY OF THE INVENTION

The present invention is directed to methods of treating pain associated with osteoarthritis of a joint in a mammal, preferably a human, wherein the methods comprise administering, preferably periodically administering, to the affected joint of the mammal a topical pharmaceutical composition comprising one or more excipients and a therapeutically effective amount of a spiro-oxindole compound.

Accordingly, one aspect of the invention is a method of treating pain associated with osteoarthritis of a joint in a mammal, wherein the method comprises administering, preferably periodically administering, to the affected joint of the mammal a topical pharmaceutical composition comprising one or more excipients and a therapeutically effective amount of a spiro-oxindole compound having the following formula:

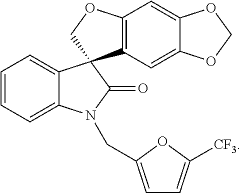 [SMILES: O=C1N(Cc2ccc(C(F)(F)F)o2)c2ccccc2[C@]12COc1cc3c(cc12)OCO3]

Another aspect is a method of locally treating pain associated with osteoarthritis of a joint in a mammal with a minimal or negligible systemic exposure, wherein the method comprises increasing the concentration of the spiro-oxindole compound defined above to a therapeutically effective amount in the synovial membrane of the affected joint in the mammal by administering, preferably periodically administering, to the affected joint a topical pharmaceutical composition comprising one or more excipients and a therapeutically effective amount of the spiro-oxindole compound.

Another aspect is a method of treating pain associated with osteoarthritis of a joint in a mammal, wherein the method comprises administering, preferably periodically administering, to the affected joint of the mammal a topical pharmaceutical composition comprising one or more excipients and a therapeutically effective amount of the spiro-oxindole compound and a therapeutically effective amount of one or more other therapeutic agents.

Specific embodiments of these aspects of the invention are described in more detail below.

### BRIEF DESCRIPTION OF THE DRAWING

FIG. 1 shows the Lund and Browder method of estimating body surface area (BSA).

### DETAILED DESCRIPTION OF THE INVENTION

As described above in the Summary of the Invention, the present invention is directed to methods of treating pain associated with osteoarthritis of a joint in a mammal, preferably a human, wherein the methods comprise administering, preferably periodically administering, to the affected joint of the mammal a topical pharmaceutical composition comprising one or more excipients and a therapeutically effective amount of a spiro-oxindole compound. These methods provide minimal to negligible systemic exposure while effectively reducing the severity of the pain or alleviating the pain due to the increased concentration of the spiro-oxindole compound in the synovial membrane of the affected joint after the administration of the pharmaceutical composition to the affected joint.

### DEFINITIONS

Unless defined otherwise in the specification, the following terms and phrases shall have the following meaning:

“A spiro-oxindole compound” or “the spiro-oxindole compound” refers to the compound having the following formula:

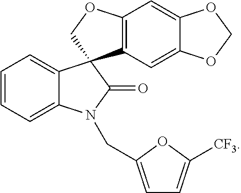 [SMILES: O=C1N(Cc2ccc(C(F)(F)F)o2)c2ccccc2[C@]12COc1cc3c(cc12)OCO3]

A chemical name for this compound is 1′-{[5-(trifluoromethyl)furan-2-yl]methyl}spiro[furo[2,3-f][1,3]benzodioxole-7,3′-indol]-2′(1′H)-one.

“Active ingredient” refers to the substance in a pharmaceutical composition which is biologically active. The active ingredient in the pharmaceutical compositions utilized in the methods of the invention is the spiro-oxindole compound.

“Adverse Event” refers to any untoward medical occurrence in a patient administered a pharmaceutical product, regardless of whether it has a causal relationship with the treatment. An adverse event can therefore be any unfavorable and unintended physical sign, symptom or laboratory parameter that develops or worsens in severity during the course of the study, or significant worsening of the disease under study or of any concurrent disease, whether or not considered related to the study drug. A new condition or the worsening of a pre-existing condition will be considered an adverse event. Stable chronic conditions (such as arthritis) that are present before study entry and do not worsen during the study will not be considered adverse events.

“Affected joint” refers to a bone joint in a mammal, preferably a human, having osteoarthritis and can include any bone joint in the body where cartilage is present. In general, an affected joint includes the shoulder joints, the spine, the joints in a hand, the joints in a foot, including the ankle, and the large weight-bearing joints, such as a knee or a hip.

The term “about” when placed before a numerical value “X” herein refers to an interval extending from X minus 10% of X to X plus 10% of X and preferably to an interval extending from X minus 5% of X to X plus 5% of X.

The expression “% w/w” refers to a percentage by weight compared to the total weight of the composition being considered.

“Baseline” refers to that information which is gathered at the beginning of a study from which variations found in the study are measured. Baseline can also be described as a known value or quantity with which an unknown is compared when measured or assessed.

“Osteoarthritis” refers to a chronic type of arthritis characterized by the breakdown of cartilage, the hard, slippery tissue that covers the ends of bones where they meet to form a joint. Unlike some other forms of arthritis, such as rheumatoid arthritis, osteoarthritis affects only joint function and does not affect skin tissue, the lungs, the eyes or the blood vessels. Healthy cartilage in a joint allows bones to glide over one another and also absorbs energy from the shock of physical movement. In osteoarthritis, the surface layer of cartilage breaks down and wears away. This breakdown of the cartilage allows the bones under the cartilage to rub together, causing pain, swelling, and loss of motion of the joint. Over time, the joint may lose its normal shape. Also, small deposits of bone, called osteophytes or bone spurs, may grow on the edges of the joint. Bits of bone or cartilage can break off and float inside the joint space. This causes more pain and damage to the joint and can cause stiffness and pain that make it difficult for a person having osteoarthritis to use that joint. Osteoarthritis can also damage ligaments and menisci. Over time osteoarthritis may create a need for joint replacements.

There are two recognized types of osteoarthritis. Primary osteoarthritis is generally associated with aging and the “wear and tear” of life. Secondary osteoarthritis, in contrast, tends to develop relatively early in life, typically 10 or more years after a specific cause, such as an injury or obesity.

Osteoarthritis occurs most often in the knees, hips and hands. Other joints, particularly the shoulders, can also be affected. Osteoarthritis rarely affects other joints, except as a result of injury or unusual physical stress.

“Pain associated with osteoarthritis of a joint” refers to the pain caused by osteoarthritis in a joint of a mammal, preferably in a joint of a human. The pain is perceived by the mammal to emanate from the joint and the tissues surrounding the joint.

“Excipient” includes, without limitation, any inactive material that is combined with a spiro-oxindole compound of the invention in order to produce a pharmaceutical composition of the invention for topical administration. The term “excipient” is intended to include, but is not limited to, any solvents, penetration enhancing agents, antioxidants, stiffening agents (i.e., thickeners), ointment bases, antioxidants, adsorbents, demulcents, emollients, preservatives, moisturizers, buffers, adjuvants, carriers, diluents, dye/colorants, solubilizers (including surfactants), wetting agents, dispersing agents, suspending agents, sunscreen agents and stabilizers. “Pharmaceutically acceptable excipient” refers to an excipient, as defined above, which has been approved by a regulatory agency, such as for example, but is not limited to, the United States Food and Drug Administration, the European Medicines Agency or Health Canada, as being acceptable for use in a formulation for the topical administration of a pharmacologically active ingredient, and/or are considered as Generally Recognized As Safe materials (GRAS materials), and/or are listed in the Inactive Ingredients Guide published by the United States Food and Drug Administration. “Pharmaceutically acceptable excipients” can also comprise the acceptable excipients listed in Remington: The Science and Practice of Pharmacy, Fox, 21^{st} ed. 2005. Exemplary excipients include, but are not limited to, the following: ascorbic acid and esters;
benzyl alcohol;
benzyl benzoate;
butylated hydroxytoluene (“BHT”);
butylated hydroxyanisole (“BHA”);
caprylic/capric triglyceride;
cetyl alcohol;
chelating agents (e.g., EDTA and citric acid);
cholesterol;
cross-linked acrylic acid based polymers (e.g., Carbopol®);
decyl methyl sulfoxide;
diethyl sebacate;
dimethylamine (“DMA”);
dimethicone;
dimethyl sulfoxide;
diethylene glycol monoethyl ether (e.g., Transcutol® P);
diisopropyl adipate (e.g., Ceraphyl® 230);
ethanol;
flavinoid;
glutathione;
glycerine;
glycerol oleate/propylene glycol (e.g., Arlacel 186);
glycerol monooleate;
polyoxyl glycerides (glyceryl caprylate/caprate and PEG-8 (polyethylene glycol) caprylate/caprate complex; carpylocaproyl macrogolglycerides (e.g., Labrasol®));
glyceryl monocaprylate (e.g., Capmul® MCM C8);
glyceryl monolinoleate (e.g., Maisine™ 35-1);
glyceryl monooleate (e.g., Peceol™);
glyceryl monostearate;
hexylene glycol;
hydroxypropyl-RP-cyclodextrin (HP-3-CD);
isopropyl alcohol;
isopropyl myristate;
laurocapram; (e.g., Azone®);
lauroyl macrogol-32 glycerides (e.g. Gelucire® 44/14);
macrogol-15 hydroxystearate (e.g., Solutol® HS15);
medium chain triglycerides (e.g., Miglyol® 810, Miglyol® 840 or Miglyol® 812);
methyl laurate;
N-methyl-2-pyrrolidine (e.g., Pharmasolve®);
mineral oil;
mono diglycerides (e.g., Capmul® MCM);
octyldodecanol;
oleic acid;
oleyl alcohol;
peanut oil;
1,2-pentanediol;
polysorbates (e.g., Tween® 80);
polyethylene glycol (e.g., PEG-8, PEG 400, PEG1000, PEG 3350, PEG 6000, or Lutrol® E 400);
polyoxyl 35 castor oil (e.g., Cremophor® EL);
polyoxyl 40 hydrogenated castor oil (e.g., Cremophor® 0 40);
propylene glycol;
propylene glycol diacetate;
propylene glycol laurate;
propylene glycol monocaprylate (e.g., Capmul PG-8, Capryol 90);
propylene glycol monolaurate (e.g., Capmul PG-12);
propylene glycol monooleate;
2-pyrrolidone;
soybean oil;
stearyl alcohol;
sulfobutylether-β-cyclodextrin (e.g., Capitsol®);
tocopherols (e.g., Vitamin E acetate);
α-tocopherol polyethylene glycol succinate (TPGS);
water; and
white petrolatum.

“Periodically administering” or “periodic administration” as used herein refers to the initial application of a topical pharmaceutical composition of the invention to the skin covering and/or surrounding a joint having osteoarthritis and then subsequent applications at pre-determined periods of time after the initial application to reduce the severity of the pain and/or to alleviate the pain associated with the osteoarthritis.

“Systemic effect” refers to a medical treatment effect that affects the body as a whole, rather than just one part.

“Minimal or negligible systemic exposure” refers to an insignificant concentration of the spiro-oxindole compound in the plasma and tissues of a mammal when compared to the concentration of the spiro-oxindole compound in the affected joint tissues after the administration of a pharmaceutical composition of the invention comprising the spiro-oxindole to the affected joint. The affected joint tissues include the skin tissue covering the affected joint, the muscle and nerve tissues within the affected joint and the synovial fluid and membrane tissue within the affected joint. For purposes of this invention, minimal or negligible systemic exposure occurs when the concentration of the spiro-oxindole compound in the plasma and tissues of the mammal, excluding the skin tissue covering the affected joint, the muscle and nerve tissues of the affected joint and the synovial fluid and membrane tissue of the affected joint, is from about 5-fold to about 100-fold less, preferably from about 5-fold to about 50-fold less, more preferably from about 10-fold to about 40-fold less, even more preferably from about 15-fold to about 25-fold less, and most preferably about 20-fold less than the concentration of the spiro-oxindole compound in the skin tissue covering the affected joint, the muscle and nerve tissue of the affected joint and the synovial fluid and membrane tissue of the affected joint after adminstering a pharmaceutical composition of the invention comprising the spiro-oxindole compound to the affected joint.

“Therapeutically effective amount” as used herein refers to that amount of a topical pharmaceutical composition of the invention or that amount of the spiro-oxindole compound in the topical pharmaceutical compositions of the invention, when administered, preferably periodically administered, to a mammal, preferably a human, is sufficient to effect treatment, as defined below, of pain associated with osteoarthritis of a joint in the mammal and with minimal or negligible systemic exposure of the spiro-oxindole compound to the mammal. The amount of the topical pharmaceutical composition of the invention or the spiro-oxindole compound which constitutes a “therapeutically effective amount” will vary depending on the nature of the pain and its severity, other conditions (e.g., sex, age, weight, general health) affecting the health of the human to be treated, and the manner of administration, as well as upon the effectiveness of the pharmaceutical composition used, but can be determined routinely by one of ordinary skill in the art having regard to his own knowledge and to this disclosure.

“Treating” or “treatment” as used herein refers to the treatment of pain associated with osteoarthritis of a joint in a mammal, preferably a human, having pain associated with osteoarthritis of a joint and includes reducing the severity of the pain and/or alleviating the pain for a period of time following the administration, preferably periodic administration of a topical pharmaceutical composition of the invention.

As described above in the Summary of the Invention, the present invention provides methods of treating pain associated with osteoarthritis of a joint in a mammal, preferably a human, wherein the methods comprise administering, preferably periodically administering, to the affected joint of the mammal a topical pharmaceutical composition comprising one or more excipients and a therapeutically effective amount of a spiro-oxindole compound. These methods provide excellent penetration of the spiro-oxindole compound into the synovial membrane of the affected joint, thereby providing a localized reduction of the severity of the pain or alleviation of the pain with minimal or negligible systemic exposure.

The topical pharmaceutical compositions of the invention are suitable for application to the skin of a mammal, preferably a human, and can be in the form of an ointment, a foam, a cream, a lotion, a gel, a liniment or other spreadable semi-liquid preparation. In one embodiment, a topical pharmaceutical composition of the invention is in the form of an ointment. In another embodiment, a topical pharmaceutical composition of the invention is in the form of a cream. In another embodiment, a topical pharmaceutical composition of the invention is in the form of a liniment. In another embodiment, a topical pharmaceutical composition of the invention is in the form of a lotion. In another embodiment, a topical pharmaceutical composition of the invention is in the form of a gel. In another embodiment, a topical pharmaceutical composition of the invention is in the form of a spreadable semi-liquid preparation.

The topical pharmaceutical compositions utilized in the methods of the invention are prepared as disclosed in U.S. Published Patent Application No. 2013/30143941 A1, the relevant disclosure of which is incorporated in full by reference herein.

Accordingly, one embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein the excipients are selected from one or more solvents, optionally from one or more penetration enhancing agents, optionally from one or more stiffening agents, optionally from one or more ointment bases, and optionally one or more antioxidants.

Another embodiment of the topical pharmaceutical compositions utilized in the methods of the invention is a topical pharmaceutical composition comprising a therapeutically effective amount of the spiro-oxindole compound, and two or more excipients, preferably pharmaceutically acceptable excipients, wherein the excipients are selected from one or more solvents, optionally from one or more penetration enhancing agents, optionally from one or more stiffening agents, optionally from one or more ointment bases, and optionally from one or more antioxidants.

Another embodiment of the topical pharmaceutical compositions utilized in the methods of the invention is a topical pharmaceutical composition comprising a therapeutically effective amount of the spiro-oxindole compound, and two or more excipients, preferably pharmaceutically acceptable excipients, wherein the excipients are selected from one or more solvents, from one or more penetration enhancing agents, from one or more stiffening agents, from one or more ointment bases, and optionally from one or more antioxidants.

Another embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein one of the excipients is a solvent selected from polyethylene glycol, diethylene glycol monoethyl ether, polysorbates, alcohols, carpylocaproyl macrogolglycerides, caprylic/capric triglyceride, fatty acid esters, diethyl sebacate, propylene glycol monocaprylate, propylene glycol laurate, mono diglycerides, glyceryl monocaprylate, medium chain triglycerides, hexylene glycol, glyceryl monooleate, 1,2-pentanediol, octyldodecanol, glyceryl mono-linoleate, glycerol oleate/propylene glycol, mineral oil, water, or glycerine.

Another embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein one of the excipients is a penetration enhancing agent selected from polyoxyl glycerides (Labrasol®), ethanol, propylene glycol laurate, diethyl sebacate, dimethyl sulfoxide, decylmethylsulfoxide, laurocapram, pyrrolidones, surfactants, alcohols, oleic acid, polyethylene glycol, diethylene glycol monoethyl ether, fatty acid esters or Transcutol® P.

Another embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein one of the excipients is a stiffening agent selected from stearyl alcohol, carbopols, dimethicone or polymers.

Another embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein one of the excipients is an ointment base selected from polyethylene glycols.

Another embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein one of the excipients is optionally an antioxidant selected from butylated hydroxytoluene, butylated hydroxyanisole, tocopherols, flavinoid, glutathione, ascorbic acid and esters, dimethyl sulfoxide, or chelating agents.

Another embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein each excipient is present in a concentration of from about 0.01% w/w to about 99% w/w.

Another embodiment of the topical pharmaceutical compositions utilized in the methods of the invention is a topical pharmaceutical composition comprising a therapeutically effective amount of the spiro-oxindole compound, and one or more excipients, preferably pharmaceutically acceptable excipients, wherein a first excipient is a solvent present at a concentration of from about 30% w/w to about 70% w/w, a second excipient is a penetration enhancing agent present in a concentration of from about 2% w/w to about 25% w/w, a third excipient is a penetration enhancing agent present in a concentration of from about 1% w/w to about 10% w/w, a fourth excipient is a penetration enhancing agent present in a concentration of from about 1% w/w to about 25% w/w, a fifth excipient is a stiffening agent present in a concentration of from about 0.1% w/w to about 10% w/w, a sixth excipient is an antioxidant present in a concentration of from about 0.01% w/w to about 2% w/w, and a seventh excipient is an ointment base present in a concentration of from about 10% w/w to about 50% w/w.

Another embodiment of the topical pharmaceutical compositions utilized in the methods of the invention is a topical pharmaceutical composition comprising a therapeutically effective amount of the spiro-oxindole compound, and one or more excipients, wherein a first excipient is a solvent present at a concentration of from about 45% w/w to about 55% w/w, a second excipient is a penetration enhancing agent present in a concentration of from about 5% w/w to about 15% w/w, a third excipient is a penetration enhancing agent present in a concentration of from about 2.5% w/w to about 7.5% w/w, a fourth excipient is a penetration enhancing agent present in a concentration of from about 2.5% w/w to about 7.5% w/w, a fifth excipient is a stiffening agent present in a concentration of from about 0.1% w/w to about 7.5% w/w, a sixth excipient is optionally an antioxidant present in a concentration of from about 0.05% w/w/ to about 1% w/w, and a seventh excipient is an ointment base present in a concentration of from about 15% w/w to about 30% w/w.

Another embodiment of the topical pharmaceutical compositions utilized in the methods of the invention is a topical pharmaceutical composition comprising a therapeutically effective amount of the spiro-oxindole compound, and one or more excipients selected from a solvent selected from PEG 400 or PEG 3350; one or more penetration enhancing agents selected from Transcutol® P, oleyl alcohol or isopropyl myristate; a stiffening agent selected from stearyl alcohol; an ointment base selected from PEG 400 or PEG 3350; and optionally an antioxidant selected from butylated hydroxytoluene.

Of this embodiment, a further embodiment is a topical pharmaceutical composition comprising a therapeutically effective amount of the spiro-oxindole compound, wherein PEG 400 is present in a concentration from about 30% w/w to about 70% w/w, Transcutol® P is present in a concentration from about 2% w/w to about 25% w/w, oleyl alcohol is present in a concentration from about 1% w/w to about 10% w/w, isopropyl myristate is present in a concentration from about 1% w/w to about 25% w/w, stearyl alcohol is present in a concentration from about 0.1% w/w to about 10% w/w, BHT is optionally present in a concentration from about 0.01% w/w to about 2% w/w, and PEG 3350 is present in a concentration from about 10% w/w to about 50% w/w.

Of this embodiment, a further embodiment is a topical pharmaceutical composition comprising a therapeutically effective amount of the spiro-oxindole compound, wherein PEG 400 is present in a concentration from about 45% w/w to about 55% w/w, Transcutol® P is present in a concentration from about 5% w/w to about 15% w/w, oleyl alcohol is present in a concentration from about 2.5% w/w to about 7.5% w/w, isopropyl is myristate present in a concentration from about 2.5% w/w to about 7.5% w/w, stearyl alcohol is present in a concentration from about 0.1% w/w to about 7.5% w/w, BHT is optionally present in a concentration from about 0.05% w/w to about 1% w/w, and PEG 3350 is present in a concentration from about 15% w/w to about 30% w/w.

Of all of the above embodiments, a further embodiment is wherein the spiro-oxindole compound is present in the topical pharmaceutical composition at a concentration from about 0.1% w/w to about 10% w/w.

Of this embodiment, a further embodiment is wherein the spiro-oxindole compound is present in the topical pharmaceutical composition at a concentration from about 2% w/w to about 8% w/w.

### Test Pharmaceutical Compositions of the Invention

In one embodiment of the invention, the topical pharmaceutical compositions utilized in the methods of the invention comprise 2% to 8% (w/w) of the spiro-oxindole compound; 45% to 55% (w/w) PEG 400; 5% to 15% (w/w) Transcutol® P; 2.5% to 7.5% (w/w) oleyl alcohol; 2.5% to 7.5% (w/w) isopropyl myristate; 0.1% w/w to 7.5% (w/w) stearyl alcohol; 0.05% to 1% (w/w) butylated hydroxytoluene; and 15% to 30% (w/w) PEG 3350. Preferably, the topical pharmaceutical compositions are in ointment form.

Of this embodiment, a topical pharmaceutical composition utilized in the methods of the invention comprises 2.0% (w/w) of the spiro-oxindole compound; 52.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350. This pharmaceutical composition is referred to herein as the “2% ointment”, the “2% pharmaceutical composition of the invention” or the “2% Test Pharmaceutical Composition”.

Of this embodiment, another topical pharmaceutical composition utilized in the methods of the invention comprises 4.0% (w/w) of the spiro-oxindole compound; 50.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350. This pharmaceutical composition is referred to herein as the “4% ointment”, the “4% pharmaceutical composition of the invention” or the “4% Test Pharmaceutical Composition”.

Of this embodiment, another topical pharmaceutical composition utilized in the methods of the invention comprises 8.0% (w/w) of the spiro-oxindole compound; 46.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350. This pharmaceutical composition is referred to herein as the “8% ointment”, the “8% pharmaceutical composition of the invention” or the “8% Test Pharmaceutical Composition”.

### Spiro-Oxindole Compound

The active ingredient of the topical pharmaceutical compositions utilized in the methods of the invention is the spiro-oxindole compound having the following formula:

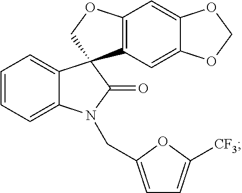 [SMILES: O=C1N(Cc2ccc(C(F)(F)F)o2)c2ccccc2[C@]12COc1cc3c(cc12)OCO3]

which is named as (S)-1′-{[5-(trifluoromethyl)furan-2-yl]methyl}spiro[furo[2,3-f][1,3]benzodioxole-7,3′-indol]-2′(1′H)one. This spiro-oxindole compound is prepared as disclosed in U.S. Pat. No. 8,450,358, the relevant disclosure of which is incorporated by reference in full.

The above spiro-oxindole compound is a potent, voltage-gated sodium channel (Na_{V}) blocker developed for the treatment of various pain indications, including neuropathic and nociceptive pain. The block of certain neuronal Na_{V}s has been demonstrated as a means for treating pain and continues to offer an approach for developing novel analgesics. In particular, the block of Na_{V}1.3, Na_{V}1.7 and Na_{V}1.8 have the most clinical and/or non-clinical evidence to date in supporting analgesic use.

Na_{V}1.3 is expressed primarily in the central nervous system in neonatal animals and at low levels throughout the body in adults (Raymond, C. K., et al., J. Biol. Chem. (2004), 279(44):46234-41). It has been demonstrated to have its expression upregulated in the dorsal horn sensory neurons of rats after nervous system injury (Hains, B. D., et al., J. Neurosci. (2003), 23(26):8881-92). Many experts in the field have considered Na_{V}1.3 as a suitable target for pain therapeutics because its expression is induced by nerve injury (Lai, J., et al., Curr. Opin. Neurobiol. (2003), (3):291-72003; Wood, J. N., et al., J. Neurobiol. (2004), 61(1):55-71; Chung, J. M., et al., Novartis Found Symp. (2004), 261:19-27; discussion 27-31, 47-54; Priest, B. T., Curr. Opin. Drug Discov. Devel. (2009) 12:682-693).

Na_{V}1.7 is expressed primarily in the peripheral nervous system in both sensory and sympathetic neurons (Raymond, C. K., et al., op. cit.). Loss-of-function mutations in SCN9A (the gene encoding the alpha subunit of the Na_{V}1.7 sodium channel) cause a human condition known as congenital indifference to pain characterized by an inability to perceive pain (Cox, J. J., et al., Nature (2006), 444:894-98; Goldberg, Y. P., et al., Clin Genet (2007), 71:311-9). Gain-of-function mutations in SCN9A are associated with inherited erythromelalgia (IEM) (Drenth, J. P., et al., J Invest Dermatology (2005), 124:1333-8), which is characterized by spontaneous or easily evoked severe pain. Furthermore, up to 29% of biopsy-proven cases of idiopathic small-fiber neuropathy manifesting mainly as distal severe pain (Faber, C. G., et al., Ann Neurol (2012), 71:26-39) are associated with gain-of-function Na_{V}1.7 channel mutations. The spiro-oxindole compound utilized in the methods of the invention displays a potent blocking effect on Na_{V}1.7. In particular, the potency of the block of the spiro-oxindole compound against Na_{V}1.7 has been shown to be approximately 220-fold greater for the inactivated state (IC₅₀=0.05 μM) compared with the resting state (IC₅₀=11.3 μM).

The expression of Na_{V}1.8 is predominately in the dorsal root ganglia (DRG) (Raymond, C. K., et al., op. cit.). The upstroke of the action potential in sensory neurons from DRG is primarily carried by current through Na_{V}1.8, so that block of this current is likely to block pain responses (Blair, N. T. and Bean, B. P., J. Neurosci. 22: 10277-90). Consistent with this finding, knock-down of Na_{V}1.8 in rats has been achieved by using antisense DNA or small interfering RNAs and virtually complete reversal of neuropathic pain was achieved in the spinal nerve ligation and chronic constriction injury models. A selective blocker of Na_{V}1.8 has been reported and it is effective at blocking both neuropathic and inflammatory pain (Jarvis, M. F. et al., Proc. Natl. Acad. Sci. USA (2007), 104 (20), 8520-5).

Topical formulations comprising the spiro-oxindole compound of the invention have demonstrated activity in a variety of nonclinical pain models including the chronic sciatic nerve constriction (chronic constriction injury [CCI]) or Bennett model of neuropathic pain, and the complete Freund's adjuvant (CFA) model of inflammatory pain.

For example, in vivo studies in neuropathic and inflammatory pain models in the rat demonstrated that topical administration of a pharmaceutical composition of the invention provided analgesic relief superior to current marketed topical therapeutics such as lidocaine and diclofenac. In the CFA model of inflammatory pain, the analgesic effects of the topical 8% pharmaceutical composition of the invention comprising the spiro-oxindole compound were obtained at a mean plasma concentration (0.32 ng/mL) that was 120-fold lower than the mean plasma concentration observed at the minimum effective dose of an oral pharmaceutical composition comprising the spiro-oxindole compound in this same model.

A dose-response was observed and the dose associated with pain relief ranged from a minimum effective dose of 2% (w/w) of the spiro-oxindole compound with the greatest effect seen at the highest dose tested of 8% (w/w) of the spiro-oxindole compound. In the CCI model of neuropathic pain, a topical 8% pharmaceutical composition exhibited a degree of efficacy comparable to 25 mg/kg of the spiro-oxindole compound by oral administration, despite systemic plasma exposure of the spiro-oxindole compound (maximum observed concentration, Cₘₐₓ=3.6 ng/mL) being significantly lower (approximately 20-fold) than that of the oral treatment group. Based on these data, the analgesic effect of a topical formulation of the spiro-oxindole compound in these animal models is considered to be due to a local effect rather than a systemic effect.

A study in minipigs was conducted to investigate the plasma pharmacokinetics, tissue distribution, and accumulation of the spiro-oxindole compound of the invention following 21 days of repeated dermal dosing, as described in more detail below in the Biological Examples. Overall, low systemic exposure was observed (mean maximum observed concentration [Cₘₐₓ]=11.7 ng/mL) with an apparent steady state achieved between Days 14 and 21. Relatively low concentrations of the spiro-oxindole compound of the invention were found in the tissues in which the spiro-oxindole compound of the invention was measured except for the skin at the drug administration site. Furthermore, in a domestic pig study, following topical administration of pharmaceutical compositions of the invention to a joint, as described in more detail below in the Biological Examples, micromolar concentrations of the spiro-oxindole compound of the invention were detected, surprisingly, in the synovial membrane tissue of the affected joint, despite very low (i.e., nanomolar) plasma concentrations. The results of this study were surprising in that they demonstrated that the spiro-oxindole compound of the invention, when topically administered, has an excellent ability to penetrate into the target joint tissue, particularly the synovial membrane of the joint, at concentration levels higher than expected with minimal or negligible systemic exposure after administration.

Without being bound by theory, the fact that the spiro-oxindole compound of the invention was previously found to be highly protein bound in human, monkey, dog, minipig, rat and mouse plasma at the 0.1, 1.0 and 10 μM concentrations (97.4% to 99.8%) may explain its ability to penetrate into joint tissue, particularly the synovial membrane of the joint, at concentration levels higher than expected with minimal or negligible systemic exposure.

Although not previously studied in patients with osteoarthritis, a topical formulation of the spiro-oxindole compound of the invention has been tested in clinical studies and was well tolerated by patients with postherpetic neuralgia (PHN) and erythromelalgia.

### Methods of the Invention

The methods of the invention are directed to the administration, preferably periodic administration, of a topical pharmaceutical composition of the invention comprising one or more excipients and a therapeutically effect amount of the spiro-oxindole compound to a mammal, preferably a human, as needed to reduce the severity of pain associated with osteoarthritis of a joint and/or to alleviate pain associated with osteoarthritis of a joint with minimal or negligible systemic exposure.

The topical pharmaceutical compositions of the invention may be administered as one-time single dose. Preferably, the topical pharmaceutical compositions of the invention are periodically administered. Typically, a periodic administration of a topical pharmaceutical composition of the invention will include an initial application of a topical pharmaceutical composition of the invention followed by a pre-determined time period and then the topical pharmaceutical composition of the invention is applied a second time to the same area and then followed by the same pre-determined time period and so forth for a specified duration of time. The pre-determined time period can be from about 4 hours to about 24 hours. In general, a topical pharmaceutical composition utilized in the methods of the invention is periodically administered to a mammal, preferably a human, having pain associated with osteoarthritis of a joint to the skin surrounding the affected joint once (qd), twice (bid), three (tid) or four (qid) times a day as needed to reduce the severity of the pain and/or to alleviate the pain. In one embodiment, a pharmaceutical composition of the invention is periodically administered once a day (every 24 hours) as needed (i.e., an effective amount of the pharmaceutical composition is topically applied to the skin surrounding the joint when the pain associated with osteoarthritis of a joint is present). In another embodiment, the pharmaceutical composition is topically administered twice a day (every 12 hours) as needed. In another embodiment, the pharmaceutical composition is topically administered three times a day (every 8 hours) as needed. In another embodiment, the pharmaceutical composition is topically administered four times a day (every 6 hours) as needed. Preferably, the pharmaceutical composition is topically administered once or twice a day.

The specified duration of time for the periodic administration of a topical pharmaceutical composition of the invention is intended to be as long as needed to alleviate or substantially relieve the pain. Preferably, the duration of the periodic administration of a topical pharmaceutical compositions of the invention is from about 1 week to about 6 months, more preferably from about 1 month to 4 months, even more preferably about 3 months.

In general, the dose volume of a pharmaceutical composition utilized in the methods of the invention which is topically administered to the area of skin surrounding the affected joint of the mammal, preferably a human, is from about 1.0 μL/cm² to about 9.0 μL/cm², preferably from about 1.0 μL/cm² to about 4.0 μL/cm² of skin, more preferably about 3.0 μL/cm².

The therapeutically effective amount of each dose of a topical pharmaceutical composition of the invention is from about 500 mg to about 2000 mg, preferably from about 500 mg to about 1500 mg, more preferably from about 750 mg to about 1200 mg, most preferably about 1200 mg per each administration to the affected joint.

The therapeutically effective amount of the spiro-oxindole compound in each dose of a topical pharmaceutical composition of the invention is from about 10 mg to about 160 mg, preferably about 24 mg in a 2% pharmaceutical composition of the invention at 3.0 μL/cm² dose volume, 48 mg in a 4% pharmaceutical composition of the invention at 3.0 μL/cm² dose volume, or 96 mg in a 8% pharmaceutical composition of the invention at 3.0 μL/cm² dose volume.

Topical administration of a pharmaceutical composition of the invention can be effected by any method commonly known to those skilled in the art. These methods include, but are not limited to, incorporation of a pharmaceutical composition of the invention into foams, creams, gels, ointments, liniments, transdermal patches or other topical formulations and delivery systems.

Topical administration of a pharmaceutical composition of the invention may be performed by a medical professional or by the patient. In certain embodiments, for maximum effectiveness and increased absorption, the area to which the pharmaceutical composition of the invention is to be administered is first cleansed, for example using an astringent, such as a standard commercial antiseptic or alcohol, or water, preferably sterile water. The area is then allowed to dry, and the pharmaceutical composition of the invention is applied onto the target area and rubbed until all the pharmaceutical composition has been absorbed or no residue remains on the skin.

The recipients of topical administration of a pharmaceutical composition of the invention can be any vertebrate animal, such as mammals. Among mammals, the preferred recipients are mammals of the Orders Primate (including humans, apes and monkeys), Arteriodactyla (including horses, goats, cows, sheep, and pigs), Rodenta (including mice, rats, rabbits, and hamsters), and Carnivora (including cats, and dogs). Among birds, the preferred recipients are turkeys, chickens and other members of the same order. The most preferred recipients are humans.

### EMBODIMENTS OF THE INVENTION

The following embodiments of the Invention are in addition to or inclusive of the embodiments disclosed above.

As noted above in the Summary of the Invention, one aspect of the invention is a method of treating pain associated with osteoarthritis of a joint in a mammal, wherein the method comprises administering, preferably periodically administering, to the affected joint of the mammal a topical pharmaceutical composition comprising one or more excipients and a therapeutically effective amount of the spiro-oxindole compound described above in the Summary of the Invention.

In one embodiment of this aspect, the method results in minimal or negligible systemic exposure of the spiro-oxindole compound.

In another embodiment of this aspect, the method results in a greater concentration of the spiro-oxindole compound in the synovial membrane and synovial fluid of the affected joint than the concentration of the spiro-oxindole compound in the plasma of the mammal.

As noted above in the Summary of the Invention, another aspect of the invention is a method of locally treating pain associated with osteoarthritis of a joint in a mammal with a minimal or negligible systemic exposure, wherein the method comprises increasing the concentration of the spiro-oxindole compound described above in the Summary of the Invention to a therapeutically effective amount in the synovial membrane of the affected joint in the mammal by administering, preferably periodically administering, to the affected joint a topical pharmaceutical composition comprising one or more excipients and a therapeutically effective amount of the spiro-oxindole compound.

In one embodiment of both aspects of the invention described above, the osteoarthritis is primary osteoarthritis.

In one embodiment of both aspects of the invention described above, the osteoarthritis is secondary osteoarthritis.

In one embodiment of both aspects of the invention described above, the pharmaceutical composition comprises 2% to 8% (w/w) of the spiro-oxindole compound.

In one embodiment of both aspects of the invention described above, the pharmaceutical composition comprises 2% to 8% (w/w) of the spiro-oxindole compound; 45% to 55% (w/w) PEG 400; 5% to 15% (w/w) Transcutol® P; 2.5% to 7.5% (w/w) oleyl alcohol; 2.5% to 7.5% (w/w) isopropyl myristate; 0.1% w/w to 7.5% (w/w) stearyl alcohol; 0.05% to 1% (w/w) butylated hydroxytoluene; and 15% to 30% (w/w) PEG 3350.

In one embodiment of both aspects of the invention described above, the pharmaceutical composition comprises 2.0% (w/w) of the spiro-oxindole compound; 52.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350.

In one embodiment of both aspects of the invention described above, the pharmaceutical composition comprises 4.0% (w/w) of the spiro-oxindole compound; 50.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350.

In one embodiment of both aspects of the invention described above, the pharmaceutical composition comprises 8.0% (w/w) of the spiro-oxindole compound; 46.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350.

In one embodiment of both aspects of the invention described above, the periodic administration is once a day, twice a day, three times a day or four times a day.

In one embodiment of both aspects of the invention described above, the periodic administration is twice a day.

In one embodiment of both aspects of the invention described above, the periodic administration is once a day.

In one embodiment of both aspects of the invention described above, the topical pharmaceutical composition is administered to the skin over and surrounding the affected joint in a dose volume of from about 1.0 μL/cm² to about 9.0 μL/cm².

In one embodiment of both aspects of the invention described above, the topical pharmaceutical composition is administered to the skin over and surrounding the affected joint in a dose volume of from about 1.0 μL/cm² to about 4.0 μL/cm².

In one embodiment of both aspects of the invention described above, the topical pharmaceutical composition is administered to the skin over and surrounding the affected joint in a dose volume of 3.0 μL/cm²

In one embodiment of both aspects of the invention described above, the therapeutically effective amount of a topical pharmaceutical composition of the invention is from about 500 mg to about 2000 mg per each administration, preferably each periodic administration, to the affected joint.

In one embodiment of both aspects of the invention described above, the therapeutically effective amount of a topical pharmaceutical composition of the invention is about 1200 mg per each administration, preferably each periodic administration, to the affected joint.

In one embodiment of both aspects of the invention described above, the therapeutically effective amount of the topical pharmaceutical composition of the invention is effective in reducing the severity of the pain or alleviating the pain.

In one embodiment of both aspects of the invention described above, the affected joint is a knee.

In one embodiment of both aspects of the invention described above, the affected joint is a joint in the hand.

In one embodiment of both aspects of the invention described above, the affected joint is a joint in a shoulder.

In one embodiment of both aspects of the invention described above, the affected joint is an ankle.

In one embodiment of both aspects of the invention described above, the affected joint is a hip.

In one embodiment of both aspects of the invention described above, the affected joint is a joint in the spine.

In one embodiment of both aspects of the invention described above, the mammal is human.

In one embodiment of both aspects of the invention described above, the administration, preferably periodic administration, of the topical pharmaceutical composition is effective in reducing an average evening pain intensity or severity in the affected joint in the human when walking on a flat surface when compared to baseline pain intensity.

In one embodiment of both aspects of the invention described above, the administration, preferably periodic administration, of the topical pharmaceutical composition is effective in reducing an average daily pain intensity or severity in the affected joint, preferably the knee or the ankle, in the human when walking on a flat surface when compared to baseline pain intensity.

In one embodiment of both aspects of the invention described above, the administration, preferably periodic administration, of the topical pharmaceutical composition is effective in reducing an average morning pain intensity or severity in the affected joint, preferably the knee or the ankle, in the human when walking on a flat surface when compared to baseline pain intensity.

In one embodiment of both aspects of the invention described above, the administration, preferably periodic administration, of the topical pharmaceutical composition of the invention is effective in increasing physical function of the affected joint during a daily activity when compared to baseline physical function.

In one embodiment of both aspects of the invention described above, the administration, preferably periodic administration, of the topical pharmaceutical composition of the invention is effective in reducing stiffness of the affected joint when compared to baseline stiffness.

In one embodiment of both aspects of the invention described above, the administration, preferably periodic administration, of the topical pharmaceutical composition of the invention is effective in reducing the intensity or severity of the pain in the affected joint as assessed by a WOMAC, PQAS-R or OMERACT-OARSI questionnaire.

In one embodiment of both aspects of the invention described above, the administration, preferably periodic administration, of the topical pharmaceutical composition of the invention is effective in reducing the intensity or severity of the pain by 30% when compared to the baseline intensity or severity of the pain.

In one embodiment of both aspects of the invention described above, the topical pharmaceutical composition of the invention is effective in reducing the intensity or severity of the pain by 50% when compared to the baseline intensity or severity of the pain.

### Combination Therapy

The methods of the invention may be usefully combined with the administration of one or more other therapeutic agents or as any combination thereof, in the treatment of pain associated with osteoarthritis of a joint. For example, the methods of the invention may utilized in the treatment of pain associated with osteoarthritis of a joint simultaneously, sequentially or separately in combination with other therapeutic methods for the administration of other agents, including, but not limited to:
opioid analgesics, e.g., morphine, heroin, cocaine, oxymorphine, levorphanol, levallorphan, oxycodone, codeine, dihydrocodeine, propoxyphene, fentanyl, hydrocodone, hydromorphone, meripidine, methadone, buprenorphine, butorphanol, nalbuphine and pentazocine;
opioid analgesics in combination with opioid antagonists, e.g., nalorphine, naloxone, naltrexone and nalmefene;
non-opioid analgesics, e.g., acetaminophen, and salicylates (e.g., aspirin);
nonsteroidal antiinflammatory drugs (NSAIDs), e.g., ibuprofen (Advil®), naproxen, fenoprofen, ketoprofen, diclofenac, diflusinal, etodolac, fenbufen, flufenisal, flurbiprofen, indomethacin, ketorolac, meclofenamic acid, mefenamic acid, meloxicam, nabumetone, nimesulide, nitroflurbiprofen, olsalazine, oxaprozin, phenylbutazone, piroxicam, sulfasalazine, sulindac, tolmetin and zomepirac;
anticonvulsants, e.g., carbamazepine, oxcarbazepine, lamotrigine, gabapentin and pregabalin;
antidepressants such as tricyclic antidepressants, e.g., amitriptyline, clomipramine, despramine, imipramine, duloxetine and nortriptyline;
COX-2 selective inhibitors, e.g., celecoxib, rofecoxib, parecoxib, valdecoxib, deracoxib, etoricoxib, and lumiracoxib;
alpha-adrenergics, e.g., doxazosin, tamsulosin, clonidine, guanfacine, dexmetatomidine, modafinil, and 4-amino-6,7-dimethoxy-2-(5-methane sulfonamido-1,2,3,4-tetrahydroisoquinol-2-yl)-5-(2-pyridyl) quinazoline;
barbiturate sedatives, e.g., amobarbital, aprobarbital, butabarbital, butabital, mephobarbital, metharbital, methohexital, pentobarbital, phenobartital, secobarbital, talbutal, theamylal and thiopental;
tachykinin (NK) antagonist, particularly an NK-3, NK-2 or NK-1 antagonist, e.g., (αR,9R)-7-[3,5-bis(trifluoromethyl)benzyl)]-8,9,10,11-tetrahydro-9-methyl-5-(4-methylphenyl)-7H-[1,4]diazocino[2,1-g][1,7]-naphthyridine-6-13-dione (TAK-637), 5-[[2R,3S)-2-[(1R)-1-[3,5-bis(trifluoromethylphenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy5-(trifluoromethoxy)phenyl]-methylamino]-2-phenylpiperidine (2S,3S);
paracetamol;
metabotropic glutamate receptor (mGluR) antagonists;
local anaesthetics such as mexiletine and lidocaine;
corticosteroids such as dexamethasone;
muscarinic antagonists, e.g., tolterodine, propiverine, tropsium t chloride, darifenacin, solifenacin, temiverine and ipratropium;
cannabinoids;
vanilloid receptor agonists (e.g., resinferatoxin) or antagonists (e.g., capsazepine);
topical agents (e.g., lidocaine, capsacin and resiniferotoxin);
muscle relaxants such as benzodiazepines, baclofen, carisoprodol, chlorzoxazone, cyclobenzaprine, methocarbamol and orphrenadine;
anti-histamines or H1 antagonists;
NMDA receptor antagonists;
Phosphodiesterase V inhibitors;
Tramadol®;
cholinergic (nicotinc) analgesics;
alpha-2-delta ligands;
prostaglandin E2 subtype antagonists;
leukotriene B4 antagonists; and
5-lipoxygenase inhibitors.

The one or other therapeutic agents utilized in the combination therapy of the invention may be administered to the mammal, preferably a human, by any route known to one skilled in the art, e.g., orally, topically, peripherally, intravenously, nasally, etc. and in any form.

A method of the invention may therefore be utilized in treating pain associated with osteoarthritis in a joint in a mammal by administering, preferably periodically administering, a topical pharmaceutical composition of the invention as defined above in the Summary of the Invention and one or more therapeutic agents. Preferably, the one or other therapeutic agent is a non-opiate analgesics, such as acetaminophen (e.g., TYLENOL®), and salicylates (e.g., aspirin).

### Kits for the Methods of the Invention

The present invention also provides kits (i.e., packages) for using the methods of the invention. The kits contain a pharmaceutical composition of the invention and instructions for the use of the pharmaceutical composition for treating pain associated with osteoarthritis of a joint. Preferably, a commercial kit will contain one or more unit doses of the pharmaceutical composition of the invention. It will be evident to those of ordinary skill in the art that any such composition which is light and/or air sensitive may require additional special packaging and/or instructions. For example, packaging may be used which is opaque to light, and/or sealed from contact with ambient air.

The present invention may be even better understood by reference to the Biological Examples which follow, but those skilled in the art will readily appreciate that the specific studies detailed are only illustrative of the invention as described more fully in the claims which follow thereafter.

### Biological Example 1

### Absorption, Distribution, Metabolism and Excretion Study in the Minipig after Repeated Daily Dermal Dosing of Pharmaceutical Composition of the Invention

The study was carried out on minipigs to obtain information on plasma pharmacokinetics, tissue distribution and accumulation, excretion and metabolic profile of the spiro-oxindole compound after daily dermal administration of a pharmaceutical composition of the invention for 20 consecutive days followed by a single dose of [¹⁴C]-spiro-oxindole compound Day 21.

The animals also received an intravenous dose of midazolam on Day 0 and again on Day 20 and blood samples were collected for analysis of plasma midazolam concentrations for investigation of CYP3A4 induction or inhibition.

Concentrations of radioactivity were measured in plasma, tissues and excreta by liquid scintillation analysis, concentrations of unchanged spiro-oxindole compound in plasma and selected tissues were determined by an LC-MS/MS bioanalytical method. Attempts to separate radioactive components present in plasma, liver and excreta using HPLC with radioactivity detection were unsuccessful due to low radioactivity levels in these samples.

Male Göttingen minipigs were used for this study to best complement previous preclinical studies performed with the spiro-oxindole compound. The minipig is considered a suitable species for this study.

### Objective of the Study

This study was carried out to obtain information during a 20-day period of repeated dermal administration of a pharmaceutical composition of the invention on the achieved concentrations of the spiro-oxindole compound in plasma (to indicate systemic accumulation), and after a final dermal dose (Day 21) of [¹⁴C]-spiro-oxindole compound on (i) the time-course of plasma radioactivity and spiro-oxindole compound concentrations, (ii) the distribution of radioactivity in body tissues, including distribution into skin layers and of unchanged drug in liver, heart, fat and skin, (iii) the rates and routes of excretion of radioactivity, (iv) the metabolite profiles in plasma, urine, bile, faeces and selected tissues, (v) the chemical nature of metabolites. The animals also received an intravenous dose of midazolam on Day 0 and again on Day 20 and blood samples were collected for analysis of plasma midazolam concentrations.

### Study Design

Midazolam was administered on Days 0 and 20 to 3 male minipigs by bolus intravenous injection at a dose level 0.5 mg/kg. On Day 1, a 8% test pharmaceutical composition of the invention was administered topically as an ointment to a clean shaven area of 209-259 cm² (6% of total body surface area) on the back of the male minipigs at a dose level of about 70 mg ointment/kg. The 8% test pharmaceutical composition comprised the following:

8% Test Pharmaceutical Composition

8.0% (w/w) of the spiro-oxindole compound (active ingredient);

46.9% (w/w) PEG 400 (solvent and ointment base);

10% (w/w) Transcutol® P (penetration enhancing agent);

5% (w/w) oleyl alcohol (penetration enhancing agent);

5% (w/w) isopropyl myristate (penetration enhancing agent);

5% (w/w) stearyl alcohol (ointment stiffening agent);

0.1% (w/w) butylated hydroxytoluene (antioxidant); and

20% (w/w) PEG 3350 (ointment base).

Administration of the 8% test pharmaceutical composition of the invention was repeated for a further 19 days and on Day 21 [¹⁴C]-spiro-oxindole composition was administered topically as an ointment for a single dose at a dose level of ca 70 mg spiro-oxindole compound equivalents ointment/kg onto the same dose site area of skin. The [¹⁴C]-spiro-oxindole composition was prepared as follows:

A suitable aliquot of [¹⁴C]-spiro-oxindole ethanol solution (0.3 mL) was transferred to a weighed amount of non-radiolabelled 8% test pharmaceutical composition ointment (2.2 g). The volume of ethanol added was ca 14% of the total volume to give a target concentration of 8% spiro-oxindole compound/g ointment. The dosing solution was continuously mixed with a spatula for approximately 30 minutes prior to determination of radioactivity concentration and weighing the final dose to each animal, and was stored overnight at room temperature

Male Gottingen minipigs were used in this study to best complement previous pre-clinical studies performed with the spiro-oxindole compound.

Concentrations of radioactivity were measured in plasma, tissues and excreta by liquid scintillation analysis, concentrations of unchanged drug in plasma and selected tissues were determined by an LC-MS/MS bioanalytical method. Attempts to separate radioactive components using HPLC with radioactivity detection present in plasma, liver and excreta were unsuccessful due to low radioactivity levels in samples submitted for further analysis.

### Experimental Procedure

### A. Materials

[¹⁴C]-spiro-oxindole compound (specific activity 55 mCi/mmol≡128 μCi/mg) was supplied as a solution and stored at ca −20° C. in the absence of moisture and light. Non-radiolabelled spiro-oxindole compound was supplied as a solid and stored at room temperature. Non-radiolabelled 8% test pharmaceutical composition of the invention as an ointment was supplied and stored at room temperature. The radiochemical purity of [¹⁴C]-spiro-oxindole compound was determined by high performance liquid chromatography (HPLC) with on-line radioactivity detection at the study center. As the radiochemical purity of the test substance provided was less then 97%, it was repurified at the study center prior to the start of the study and stored at ca −20° C. in the absence of moisture and light. The radiochemical purity of the repurified batch was 99.2% by HPLC prior to the start of the study.

All other chemicals used in this study were reagent grade or analytical grade, as appropriate, and were obtained from approved commercial suppliers. Purified water was produced on the premises.

### B. Animal Management

Three (3) male Ellegaard Gottingen Minipigs having no previous history of any test xenobiotic treatment were obtained for use on this study. The estimated age of these animals was 19 weeks at the time of first administration and their body weights at the time of first administration were approximately 10 kg. After arrival at the test center, the minipigs were subjected to a suitable physical examination to accepted animal husbandry procedures to ensure their suitability for inclusion in the study. The minipigs were randomly allocated individual identification in the form of ear tags and the study schedule number combined with the animal number constituted a unique identification of each animal.

The minipigs were allowed an acclimatization period of approximately 3 weeks prior to treatment with the test substance. During the acclimatization period and for the duration of the study period, the animals were group-housed in indoor pens except during the period of sample collection following intravesical dose administration when they were housed individually in stainless-steel metabolism cages equipped with wire mesh floors and plastic netting below to allow the separate collection of urine from faeces. Whilst housed within the indoor pens, the animals were provided with cereal straw bedding and soiled bedding was changed daily.

The rooms in which the minipigs were housed were well ventilated with regular air changes, and lit using artificial light, which was controlled to provide an alternating 12-hour light/dark cycle. The ambient air temperature in the animal housing unit was in the range of 15-24° C., and the relative humidity between 40% and 70%; both temperature and humidity were continuously monitored and automatically recorded.

During the course of the study, the minipigs were routinely observed for behavioural changes, and any indications of ill health or reaction to treatment. On the day of dosing, the animals were observed immediately after dosing, again within 2 hours of completion of dosing the study group/phase and on at least one other occasion towards the end of the working day. On all other days post-dose, the animals were observed on at least one occasion, i.e., during the initial animal check procedure.

### C. Dose Administration

Midazolam doses were administered by bolus intravenous administration at a target dose level of 0.5 mg/kg and were quantified using nominal concentration of midazolam provided and the volume administered to each animal.

On the day prior to the first dermal dose, the dorsum and flanks of each animal were carefully clipped using electric clippers and shaved using an electric rotary head shaver (Phillips). Clipping and shaving was as close as possible to the skin, whilst avoiding damage to the dose site. Photographs were taken to demonstrate the skin integrity on the clipped area. The corners of the treatment area (equivalent to about 6% body surface area, 11 cm×19 cm-12 cm×22 cm) were marked using an indelible pen. The process of hair removal was repeated periodically during the dose administration period as necessary.

Dosages of test formulations were applied with foil and spread using nitrile gloves to form a thin uniform layer to the treatment site. The dose site remained non-occluded during the 20 days of study composition applications (8% test pharmaceutical composition) but was semi-occluded immediately after the radiolabelled dose application on Day-21 using a tubular elastic net bandage secured in place with a dressing retention tape.

The 8% test pharmaceutical composition of the invention application was quantified from the weight of test formulation applied over the treatment area. [¹⁴C]-oxindole composition doses were applied in the same manner over the same dose site area as the 8% test pharmaceutical composition and covered with dressings. The radioactive dose administered to each animal was calculated from the weight of radiolabelled composition supplied and the measured radioactivity concentration. Any residual radiolabelled material remaining on the treatment foil was not quantified so was not included in the calculation of dose administered to each animal.

### D. Sample Collection

After intravenous administration of midazolam on Day 0, blood samples (ca 1 mL) were collected by venepuncture (not the vein used for dosing) at predose, 2, 30 minutes, and at 1, 2, 4 and 6 hours post dose and delivered into K₂EDTA anticoagulant tubes. Midazolam dosing and subsequent blood sampling were repeated on Day 20 (2 hours post dosing with the 8% test pharmaceutical composition of the invention). All midazolam blood samples were centrifuged (ca 2000×‘g’ for 10 minutes at ca 4° C.) to obtain the plasma which was transferred into clean polypropylene tubes and stored at ca −20° C.; blood cells were discarded.

Immediately prior to application of the 2^{nd}, 7^{th}, 14^{th} dose of 8% test pharmaceutical composition of the invention and immediately prior to the final dose of [¹⁴C]-spiro-oxindole composition on Day 21, a blood sample (ca 2 mL) was collected by venepuncture from each minipig and delivered into K₂EDTA anticoagulant tubes. The blood was centrifuged (ca 2000×‘g’ for 10 minutes at ca 4° C.) to obtain the plasma which was transferred into clean polypropylene tubes, divided into two portions and stored at ca −20° C.; blood cells were discarded.

At the following times after the [¹⁴C]-spiro-oxindole composition dermal application on Day 21: 0.5, 1, 2, 4, 8, 12 and 24 hours, blood (ca 3 mL) was collected by venepuncture from each minipig and delivered into K₂EDTA anticoagulant tubes. The blood was centrifuged (ca 2000×‘g’ for 10 minutes at ca 4° C.) to obtain the plasma which was transferred into clean polypropylene tubes and stored at ca −20° C. pending analysis; blood cells were discarded.

Urine was collected (into containers cooled in solid CO₂) from all minipigs overnight prior to dosing the first Midazolam dose and [¹⁴C]-spiro-oxindole composition dose, and during 0-6 and 6-24 hours postdose. Feces were collected separately overnight prior to dosing and during 0-24 hours after the [¹⁴C]-spiro-oxindole composition dose. After collection of the final excreta samples, cages were first washed with water (ca 1 Litre) and then methanol (ca 1 Litre), and the washings were retained for radioactivity analysis.

At 24 hours after application of the [¹⁴C]-spiro-oxindole composition dose, the semi-occlusive dressings were removed and the dose area cleansed with copious volumes of warm dilute soap solution and dabbed dry with the minimum amount of paper medical wipe. All dressings, washings and wipes were retained for radioactivity measurement. The animals were then sacrificed (pentobarbitone overdose), the stratum corneum at the dose site were removed by tape-stripping (10-20 strips) and retained for radioactivity analysis. The dose site was then excised from the carcass and divided into two approximately equal portions. For one portion, the epidermis was separated from the underlying dermis by transferring each skin sample to a foil boat and heating in a waterbath at 60° C. for approximately 20 minutes. The second portion was immediately attached to cork discs with embedding medium and snap frozen in isopentane cooled with liquid nitrogen. These mounted and frozen samples were stored at ca −70° C. until taken for microautoradiography.

The following tissues/organs were removed from the remaining carcass:

Adrenal gland
Aorta
Bile (from the gall bladder)
Bone
Bone marrow
Brain
CSF
Epididymis
Eye
Fat (white)
Fat (brown)
Heart
Kidney
Lacrimal gland
Liver
Lungs
Lymph nodes
Muscle (remote from dose site)
Muscle (underlying dose site)
Pancreas
Pituitary
Prostate
Salivary gland
Sciatic nerve
Synovial membrane (knee joint)
Skin (remote from the dose site)
Skin (from the dose site after washing and
tape stripping)
Spinal cord
Spleen
Testis
Thymus
Thyroid
Urinary bladder
Vena cava
Stomach wall
Stomach content
Small intestine wall
Small intestine content
Large intestine wall
Large intestine content

The urinary bladder from each animal was rinsed with saline and the washings discarded. Each section of the gastrointestinal tract wall was washed to remove contents (which were retained for analysis) prior to analysis. Remaining carcasses were discarded.

### Results

Concentrations of radioactivity were measured in plasma, tissues and excreta by liquid scintillation analysis, concentrations of unchanged spiro-oxindole compound in plasma and selected tissues were determined by an LC-MS/MS bioanalytical method. Attempts to separate radioactive components present in plasma, liver and excreta using HPLC with radioactivity detection were unsuccessful due to low radioactivity levels in these samples.

The concentration of the spiro-oxindole compound in plasma (in ng/mL) prior to Days 2, 7 and 14 of 20 daily dermal doses of the 8% test pharmaceutical composition of the invention and following 20 daily dermal doses of the 8% test pharmaceutical composition of the invention followed by a single dermal administration of [¹⁴C]-spiro-oxindole composition ointment (ca 70 mg ointment/kg/day) in male minipigs is shown below in Tables 1 and 2:

TABLE 1
PLASMA COLLECTED AFTER REPEAT ADMINISTRATION
OF 8% TEST PHARMACEUTICAL COMPOSITION
OF THE INVENTION

| | ng/mL | | | | |
|---|---|---|---|---|---|
| Time | 3M | 4M | 5M | Mean | sd |
| Predose Day 2 | 1.85 | 1.82 | 0.619 | 1.43 | 0.70 |
| Predose Day 7 | 2.89 | 1.32 | 1.28 | 1.83 | 0.92 |
| Predose Day 14 | 11.7 | 2.56 | 3.33 | 5.86 | 5.07 |
| Predose Day 21 | 4.64 | 2.94 | 2.40 | 3.33 | 1.17 |
| 24 h postdose Day 21 | 12.7 | 3.86 | 1.63 | 6.06 | 5.85 |
| (Analogous to predose | | | | | |
| Day 22) | | | | | |

TABLE 2
PLASMA COLLECTED AFTER REPEAT ADMINISTRATION
OF 8% TEST PHARMACEUTICAL COMPOSITION OINT-
MENT FOLLOWED BY A SINGLE ADMINISTRATION OF
[¹⁴C]-SPIRO-OXINDOLE COMPOSITION

| Time after administration of | | ng/mL | | | | |
|---|---|---|---|---|---|---|
| [¹⁴C]-spiro-oxindole composition | | 3M | 4M | 5M | Mean | sd |
| 30 | min | 4.11 | 1.26 | 1.63 | 2.33 | 1.55 |
| 1 | h | 2.64 | 1.67 | 2.55 | 2.29 | 0.54 |
| 2 | h | 3.11 | 1.86 | 12.7 | 5.89 | 5.93 |
| 4 | h | 4.75 | 2.97 | 4.02 | 3.91 | 0.89 |
| 8 | h | 12.4 | 19.6 | 3.15 | 11.7 | 8.3 |
| 12 | h | 5.14 | 5.03 | 3.19 | 4.45 | 1.10 |
| 24 | h | 12.7 | 3.86 | 1.63 | 6.06 | 5.85 |

sd Standard deviation

Concentrations of radioactivity in plasma following the 20 daily dermal does of the 8% test pharmaceutical composition of the invention followed by a single dermal administration of [¹⁴C]-spiro-oxindole composition (ca 70 mg ointment/kg/day) to the male minipigs is shown below in Table 3:

TABLE 3
CONCENTRATIONS OF RADIOACTIVITY IN PLASMA FOL-
LOWING 20 DAILY DERMAL DOSES OF 8% TEST PHARMA-
CEUTICAL COMPOSITION FOLLOWED BY A SINGLE
DERMAL ADMINISTRATION OF [¹⁴C]-SPIRO-OXINDOLE
COMPOUND COMPOSITION(CA 70 MG OINTMENT/KG/DAY)

ng equivalents spiro-oxindole compound/mL

| Time | 3M | 4M | 5M | Mean | sd |
|---|---|---|---|---|---|
| Plasma | | | | | |
| Predose | BLQ | BLQ | BLQ | BLQ | — |

| | | | | | | |
|---|---|---|---|---|---|---|
| 30 | min | BLQ | BLQ | BLQ | BLQ | — |
| 1 | h | BLQ | BLQ | BLQ | BLQ | — |
| 2 | h | BLQ | BLQ | BLQ | BLQ | — |
| 4 | h | BLQ | 7.03 | BLQ | BLQ | — |
| 8 | h | 7.82 | 11.7 | 10.3 | 9.94 | 1.96 |
| 12 | h | 12.6 | 15.6 | 10.5 | 12.9 | 2.6 |
| 24 | h | 17.9 | 22.3 | 15.9 | 18.7 | 3.3 |

sd Standard deviation
BLQ Below the limit of quantification (<5.55-7.40 ng equivalents spiro-oxindole compound/mL)

Concentrations of the spiro-oxindole compound in plasma were above the limit of quantification (>0.5 ng/mL) at predose on Day 2 (i.e., approximately 24 hours after a single dermal dose of 8% test pharmaceutical composition of the invention) when they measured a mean of 1.43 ng/mL, then increased steadily until predose on Day 14 to a mean of 5.86 ng/mL. At predose on Day 21, mean spiro-oxindole compound concentrations were 3.33 ng/mL, which were similar to concentrations observed on Day 14, indicating that steady-state was achieved between Days 14 and 21. Following the last dose (on Day 21), mean plasma concentrations of spiro-oxindole compound increased and were maximal at 8 hours post dose, then declined and at 24 hours post dose, mean plasma concentrations were 6.06 mg/mL, which was significantly similar to concentrations observed at predose on Day 21.

Conversely, mean plasma radioactivity concentrations were generally below the limit of quantification until 8 hours after administration of the radiolabelled ointment on Day 21 when they measured 11.7 ng/mL. Thereafter, mean plasma radioactivity concentrations increased until the final sampling time (24 hours post dose).

Following a single dermal application of [¹⁴C]-spiro-oxindole compound ointment to the male minipigs following 20 daily dermal applications of 8% test pharmaceutical composition of the invention, <1% of the administered dose was excreted during 24 hours post final dose. Approximately 33% of the administered dose was recovered following exhaustive solvent extraction of dose dressings and a further 21% recovered in dose site swabs. The proportions of the dose associated with the dressings and dose site swabs can be considered to consist entirely of unabsorbed drug. Exhaustive solvent extraction of tape strippings used to remove the stratum corneum from each animal yielded another 2% and a further 26% dose was associated with the epidermis and dermis of the excised dose site excised. The mean overall recovery of radioactivity in male minipigs was 82.36% of the administered dose.

Tissue radioactivity concentrations were generally below the limit of quantification (ca 12-25 ng equivalents spiro-oxindole compound/g) in the majority of tissues analysed. Of the tissues with radioactivity concentrations above the limit of quantification, mean radioactivity concentrations (ng equivalents spiro-oxindole compound/g) were maximal in skin from the dose site (25500), gall bladder contents (4870), fatty tissues (273 [abdominal], 100 [brown]), bone marrow (154), muscle underlying the dose site (142) and skin remote from the dose site (111). Concentrations of the spiro-oxindole compound were also measured in selected tissues and were maximal in skin from the dose site (1860 ng/g), white fat (53.8 ng/g), heart (17.5 ng/g) and liver (11.3 ng/g). The corresponding total radioactivity in these tissues were 25500, 273, 18.5 and 74.9 ng equiv spiro-oxindole compound/g, respectively.

Following UPLC analysis of urine and bile, two distinct peaks were observed in urine whilst in bile (gall bladder contents) another two peaks were observed. Radioactivity levels in these samples were too low for accurate quantification or identification but none were consistent with spiro-oxindole compound. Attempts to separate radioactive components using HPLC with radioactivity detection present in liver and excreta were unsuccessful due to low radioactivity levels in these samples.

Results of microautoradiography of skin samples removed from the dose site after the final dermal dose of [¹⁴C]-spiro-oxindole compound ointment indicated localization of spiro-oxindole compound-related material in the epidermis, dermis and hair follicles. Greatest concentrations were found at the epidermis and dermo-epidermal junction, and at the hair follicles, particularly the hair follicle bulbs.

Overall in this study, low systemic exposure of the spiro-oxindole compound was observed along with relatively low concentration of the spiro-oxindole compound in the tissues examined except for the skin from the dose site.

### Biological Example 2

### Joint Penetration and Tissue Distribution Study in Domestic Pig Following Multiple-Dose Topical Administration of Spiro-Oxindole Compound

This study was conducted to evaluate the joint penetration, systemic plasma exposure and tissue distribution of the spiro-oxindole compound in domestic pigs following topical administration of a pharmaceutical composition utilized in the methods of the invention. Fifteen female pigs were used in this study with three pigs in each group. Beginning on Day 1, a pharmaceutical composition of the invention was topically applied to the left radiocarpal joint (carpus) and tibiotarsal joint (hock) twice daily (bid) for 17 days (34 applications) for Groups 1-4 and once daily (qd) for 17 days (17 applications) for Group 5. Pre-dose blood samples (prior to morning dosing) was collected on Days 2, 7, 10, 14 and 17. A full pharmacokinetic sampling was performed on Day 17 (post am dosing) at the following time points: 1, 2, 4, 8 and 12 hr. On the day of necropsy, Day 18, blood was collected prior to euthanasia, each pig was humanely euthanized and dorsal skin and muscle, liver, kidney, heart, lung, brain, intestine, fat, ovaries, urinary bladder, sciatic nerve, and skin, muscle, synovial membrane, and synovial fluid from all 4 joints (2 treated, left carpus and hock, and 2 untreated, right carpus and hock) was collected.

### Dose

Doses were calculated by measuring the joint circumference and joint width (to include ˜2 cm above and below the joint) to calculate the total surface area for each individual dose. Group 1 received a 2% pharmaceutical composition of the invention and the dose volume for Group 1 was calculated using the total surface area and the designated dose volume of 3 μL/cm². The total dose volume was then converted from μL to mL to estimate the dose weight in grams, based on the composition density of 1 g/cm³. Following is an example (not actual measurements) of calculating the dose:

| | Total | Joint | Total | | | | |
|---|---|---|---|---|---|---|---|
| | Dose | circum- | Surface | Dose | Dose | Dose | Dose |
| | width | ference | Area | (μL/ | Volume | Volume | Weight |
| Joint | (cm)* | (cm) | (cm²)** | cm²) | (μL) | (mL) | (gm)*** |
| Left | 8.0 | 15 | 120.0 | 3 | 360 | 0.36 | 0.36 |
| carpus | | | | | | | |
| Left | 9.0 | 19 | 171.0 | 3 | 513 | 0.51 | 0.51 |
| hock | | | | | | | |

*Determined prior to dosing; joint was measured to include ~2 cm above and below the joint and a distance above and below the joint capsules was determined for dose administration.
**Joint width × joint circumference.
***Assuming 1 mL = 1 gm for dosing purposes.

For Groups 2-5, the dose volume was calculated using the total surface area and the designated dose volume of 3 μL/cm² (Groups 2, 3 and 5) or 1 μL/cm² (Group 4). The total dose volume was then converted from μL to mL to estimate the dose length in centimeters of a ribbon of the pharmaceutical composition when squeezed from a glaminate tube onto a metal metric ruler, based on the composition length-weight relationship of 1 cm of pharmaceutical composition being equal to 0.5 g of the composition (diameter of the tube opening was 0.87 cm).

### Test Pharmaceutical Compositions and Administration

The hair on the left carpus and hock of each animal was clipped approximately 3 days prior to treatment to allow for any razor burn or skin scrapes to heal before administration of a test pharmaceutical composition. The test pharmaceutical compositions were comprised as follows:

### 2% Test Pharmaceutical Composition

2.0% (w/w) of the spiro-oxindole compound (active ingredient);

52.9% (w/w) PEG 400 (solvent and ointment base);

10% (w/w) Transcutol® P (penetration enhancing agent);

5% (w/w) oleyl alcohol (penetration enhancing agent);

5% (w/w) isopropyl myristate (penetration enhancing agent);

5% (w/w) stearyl alcohol (ointment stiffening agent);

0.1% (w/w) butylated hydroxytoluene (antioxidant); and

20% (w/w) PEG 3350 (ointment base).

### 4% Test Pharmaceutical Composition

4.0% (w/w) of the spiro-oxindole compound (active ingredient);

50.9% (w/w) PEG 400 (solvent and ointment base);

10% (w/w) Transcutol® P (penetration enhancing agent);

5% (w/w) oleyl alcohol (penetration enhancing agent);

5% (w/w) isopropyl myristate (penetration enhancing agent);

5% (w/w) stearyl alcohol (ointment stiffening agent);

0.1% (w/w) butylated hydroxytoluene (antioxidant); and

20% (w/w) PEG 3350 (ointment base).

### 8% Test Pharmaceutical Composition

8.0% (w/w) of the spiro-oxindole compound (active ingredient);

46.9% (w/w) PEG 400 (solvent and ointment base);

10% (w/w) Transcutol® P (penetration enhancing agent);

5% (w/w) oleyl alcohol (penetration enhancing agent);

5% (w/w) isopropyl myristate (penetration enhancing agent);

5% (w/w) stearyl alcohol (ointment stiffening agent);

0.1% (w/w) butylated hydroxytoluene (antioxidant); and

20% (w/w) PEG 3350 (ointment base).

The 2% Test Pharmaceutical Composition was topically administered to the animals of Group 1.

The 4% Test Pharmaceutical Composition was topically administered to the animals of Group 2.

The 8% Test Pharmaceutical Composition was topically administered to the animals of Group 3, Group 4 and Group 5.

The test compositions were topically applied an equal distance above and below the center of the joint, i.e., inter-carpal joint space (carpus) and inter-tarsal joint space (hock) of each animal to allow for complete coverage of the joint, including approximately 2 cm above and below the edges of the joint. The test composition was applied to the entire circumference of the designated area. All animals were dosed using the same distance above and below each joint center for consistency.

The dose volume (μL/cm²) for the test pharmaceutical compositions was 3.0 μL/cm² for Groups 1, 2, 3 and 5 and 1.0 μL/cm² for Group 4.

### Pharmacokinetic Samples

Approximately 2 mL of whole blood was collected prior to the morning dose administration on Days 2, 7, 10, 14 and 17. A full pharmacokinetic profile sampling was performed on Day 17 after the morning dose administration at the following time points: 1, 2, 4, 8 and 12 hr. On the day of necropsy, Day 18, blood was collected prior to euthanasia.

### Tissue Collection

The following tissue samples were collected after euthanasia:
Dorsal skin and muscle (from the mid-back)
Liver—Left Medial Lobe
Kidney—Right
Heart
Lung—Right diaphragmatic lobe
Brain—Right cerebral hemisphere
Intestine—mid-jejunum
Duodenum (10-15 cm length) from all 6 animals in Groups 1 and 2
Fat
Ovary—Right
Urinary bladder
Sciatic nerve from both legs
Skin, muscle underlying site of administration and synovial membrane of all 4 joints (treated left carpus and hock and untreated right carpus and hock)

### Results

The concentration of the spiro-oxindole compound in the synovial membrane of the treated joints of the test animals is shown below in Table 4:

TABLE 4
SPIRO-OXINDOLE COMPOUND CONCENTRATION
IN SYNOVIAL MEMBRANE TISSUE

| | Untreated | | Treated | |
|---|---|---|---|---|
| | Test Animal No. | | Test Animal No. | |
| Group | and Joint | ng/g | and Joint | ng/g |
| G1 | #1 right carpus | 44.47 | #1 left carpus | 105.89 |
| | #1 right hock | 41.19 | #1 left hock | 54.04 |
| | #2 right carpus | 70.83 | #2 left carpus | 126.88 |
| | #2 right hock | 97.22 | #2 left hock | 152.13 |
| | #3 right carpus | 84.97 | #3 left carpus | 56.86 |
| | #3 right hock | 33.16 | #3 left hock | 38.86 |
| G2 | #4 right carpus | 114.15 | #4 left carpus | 72.14 |
| | #4 right hock | 92.75 | #4 left hock | 132.05 |
| | #5 right carpus | 100.42 | #5 left carpus | 269.82 |
| | #5 right hock | 69.80 | #5 left hock | 95.93 |
| | #6 right carpus | 129.30 | #6 left carpus | 183.72 |
| | #6 right hock | 98.28 | #6 left hock | 131.87 |
| G3 | #7 right carpus | 54.19 | #7 left carpus | 708.62 |
| | #7 right hock | 82.74 | #7 left hock | 252.46 |
| | #8 right carpus | 185.83 | #8 left carpus | 737.86 |
| | #8 right hock | 737.82 | #8 left hock | 576.83 |
| | #9 right carpus | 130.63 | #9 left carpus | 137.46 |
| | #9 right hock | 167.55 | #9 left hock | 1917.93 |
| G4 | #10 right carpus | 34.85 | #10 left carpus | 49.99 |
| | #10 right hock | 26.20 | #10 left hock | 291.56 |
| | #11 right carpus | 1772.86 | #11 left carpus | 59.22 |
| | #11 right hock | 0.00 | #11 left hock | 38.32 |
| | #12 right carpus | 46.92 | #12 left carpus | 159.68 |
| | #12 right hock | 45.29 | #12 left hock | 3171.10 |
| G5 | #13 right carpus | 11.61 | #13 left carpus | 702.22 |
| | #13 right hock | 18.69 | #13 left hock | 31.44 |
| | #14 right carpus | 25.25 | #14 left carpus | 150.00 |
| | #14 right hock | 37.98 | #14 left hock | 100.00 |
| | #15 right carpus | 79.58 | #15 left carpus | 35.31 |
| | #15 right hock | 21.70 | #15 left hock | 55.90 |

The concentration of the spiro-oxindole compound (in ng/mL) in plasma of the test animals is shown below in Table 5:

TABLE 5
SPIRO-OXINDOLE COMPOUND CONCENTRATION IN PLASMA

| | | | | | | Day 17 | Day 17 | Day 17 | Day 17 | Day 17 | |
|---|---|---|---|---|---|---|---|---|---|---|---|
| | Day 2 | Day 7 | Day 10 | Day 14 | Day 17 | 1 hour | 2 hour | 4 hour | 8 hour | 12 hour | |
| Animal | Before | Before | Before | Before | Before | after | after | after | after | after | |
| Number | dosing | dosing | dosing | dosing | dosing | dosing | dosing | dosing | dosing | dosing | Day 18 |
| #1 | 1.02 | 1.36 | 1.14 | 3.44 | 1.13 | 7.59 | 1.10 | 8.22 | 1.91 | 10.28 | 3.94 |
| #2 | 1.65 | 4.12 | 1.87 | 4.69 | 1.43 | 2.71 | 2.16 | 1.67 | 2.03 | 12.85 | 3.24 |
| #3 | 0.34 | 0.97 | 1.54 | 1.10 | 2.70 | 1.21 | 1.07 | 1.04 | 1.30 | 11.38 | 1.60 |
| #4 | 0.84 | 1.73 | 1.79 | 2.47 | 3.25 | 2.47 | 3.47 | 2.72 | 1.94 | 12.17 | 5.41 |
| #5 | 0.86 | 1.74 | 5.34 | 4.35 | 3.90 | 2.78 | 4.20 | 3.56 | 3.02 | 15.72 | 2.83 |
| #6 | 1.24 | 2.66 | 2.11 | 3.40 | 2.24 | 2.24 | 2.48 | 4.83 | 1.62 | 21.78 | 3.29 |
| #7 | 1.47 | 1.50 | 5.48 | 2.19 | 14.43 | 0.98 | 2.37 | 1.85 | 1.65 | 27.61 | 2.76 |
| #8 | 1.21 | 4.40 | 5.13 | 4.81 | 2.45 | 9.03 | 4.47 | 4.29 | 3.36 | 16.89 | 6.10 |
| #9 | 2.36 | 0.00 | 3.64 | 6.86 | 3.90 | 7.37 | 5.31 | 6.23 | 3.43 | 102.48 | 4.13 |
| #10 | 0.24 | 4.82 | 1.10 | 1.90 | 1.12 | 2.62 | 3.51 | 2.27 | 1.29 | 9.98 | 1.09 |
| #11 | 0.71 | 1.89 | 1.24 | 2.63 | 0.72 | 5.22 | 3.57 | 2.03 | 2.15 | 15.16 | 2.36 |
| #12 | 1.34 | 0.99 | 1.88 | 2.14 | 1.41 | 1.01 | 1.94 | 2.74 | 2.83 | 24.37 | 1.34 |
| #13 | 2.68 | 3.34 | 1.14 | 2.77 | 2.17 | 3.49 | 3.53 | 3.70 | 4.21 | 10.60 | 2.10 |
| #14 | 1.26 | 0.92 | 1.87 | 2.74 | 0.63 | 0.67 | 1.65 | 1.85 | 7.22 | 4.97 | 0.96 |
| #15 | 0.43 | 1.29 | 1.07 | 1.53 | 0.99 | 2.49 | 2.56 | 1.28 | 1.00 | 8.47 | 4.94 |

The concentration of the spiro-oxindole compound (in ng/g) in liver tissue of the test animals is shown in Table 6 below:

TABLE 6
SPIRO-OXINDOLE COMPOUND CONCENTRATION
IN LIVER TISSUE

| Group | Animal No. | ng/g |
|---|---|---|
| G1 | #1 | 17.0 |
| | #2 | 2.2 |
| | #3 | 14.0 |
| G2 | #4 | 0/0 |
| | #5 | 49.1 |
| | #6 | 29.3 |
| G3 | #7 | 47.2 |
| | #8 | 48.2 |
| | #9 | 21.6 |
| G4 | #10 | 0.0 |
| | #11 | 39.1 |
| | #12 | 13.0 |
| G5 | #13 | 3.6 |
| | #14 | 25.1 |
| | #15 | 0.0 |

The above results indicate that periodic administration of the topical pharmaceutical compositions of the invention provided an unexpectedly high concentration of the spiro-oxindole compound in the synovial membrane of the treated joints of the test animals with minimal or negligible systemic exposure of the spiro-oxindole compound to plasma or to liver tissue.

### Biological Example 3

### Study to Evaluate the Safety and Efficacy of Topically Applied Pharmaceutical Compositions of the Invention in Patients with Primary Osteoarthritis Affecting a Single Knee

This study is a randomized, double-blind, placebo-controlled clinical trial (Phase 2) to evaluate the safety and efficacy of topically applied pharmaceutical compositions of the invention to humans with primary osteoarthritis affecting a single knee.

### Study Population and Number

The study population will comprise approximately 375 patients (men and women) between the ages of 40 and 85 years old, inclusive, with primary osteoarthritis predominantly affecting a single knee. The patients (125 per treatment group) will be randomly assigned to treatment to ensure that approximately 100 patients per group complete the treatment period.

The primary osteoarthritis will be confirmed by American College of Rheumatology criteria for diagnosis of osteoarthritis (Altman R, Asch E, Bloch D, Bole G, Borenstein D, Brandt K, et al., “Development of criteria for the classification and reporting of osteoarthritis. Classification of osteoarthritis of the knee. Diagnostic and Therapeutic Criteria Committee of the American Rheumatism Association”, Arthritis Rheum 1986; 29:1039-4) and a Kellgren-Lawrence scale (Kellgren J H, Lawrence J S. Radiological assessment of osteo-arthrosis, Ann Rheum Dis 1957; 16:494-502) rating of 2 to 3 on a radiograph of the target knee as read by a central radiologist blinded to the patient's treatment group. The primary osteoarthritis pain in the target knee must be present most days in the previous 3 months. Pain in the target knee upon walking must meet specific criteria for the visual analog scale (VAS) score (Question 1 on the Western Ontario and McMaster Universities Arthritis Index [WOMAC] questionnaire).

### Study Duration

The duration of the study participation will be approximately 12 weeks consisting of a 4-week screening period, a 4-week treatment period, and a 4-week follow-up period.

### Study Drug, Dose, Mode of Administration, Area of Administration and Rate of Administration

The study drug is a double-blind 4% pharmaceutical composition of the invention or 8% pharmaceutical composition of the invention as defined herein in ointment form or placebo ointment for topical administration. The study drug will be applied (i.e., administered) twice daily (bid) to the target knee in the morning (0700±2 hours) and again in the evening (1900±2 hours) from Day 1 through Day 28. The study drug will be applied at 3 μL/cm² per application. The actual amount (mg of ointment) of the study drug per application will be measured (as cm of ointment) by a dosing card.

The pharmaceutical compositions utilized in this study will be supplied to the study centers as 50 g fills in 60-mL plastic laminate tubes with tamper-evident seals. The tubes will be stored at ambient room temperature (15° C. to 30° C.). All patients will be provided with tubes of blinded study drug ointment (4% ointment, 8% ointment, or placebo ointment) to be applied twice daily (bid) to the target knee during the 4-week treatment period.

The anticipated surface area of the knee to be covered with ointment (anterior, medial, and lateral knee surfaces) is approximately 400 cm². A study in pigs (see Biological Example 1) demonstrated that topical application of the 8% and 4% ointment at 3 μL/cm² to the skin over the knee joint produced tissue levels in synovial tissue above the levels projected to provide efficacy. Using a similar dose volume of 3 μL/cm² over a 400 cm² knee area will require 1200 mg (given the study ointment density of 1 mg/1 μL). The appropriate amount (length) of ointment will be measured using a dosing card for each application. Because each 1 cm expressed from the tubes contains 0.5 g of the spiro-oxindole compound, 2.5 cm (i.e. ˜1 inch) will be measured for each dose.

### Method of Blinding and Randomization

The study drug tubes containing the 4% and the 8% pharmaceutical composition of the invention will be identical and the ointments of the individual pharmaceutical composition group will be indistinguishable among the pharmaceutical compositions of the invention and the placebo (which will have the same composition as the pharmaceutical compositions of the invention except that the spiro-oxindole compound is not present and the amount of PEG400 in the placebo composition is increased by the amount of the spiro-oxindole compound if it were present). Patients, investigators, and all clinical study center staff will remain blinded to treatment assignment during the study. Eligible patients will be randomly assigned via interactive response technology (IRT) in a 1:1:1 ratio to receive 4% pharmaceutical composition as an ointment, 8% pharmaceutical composition as an ointment, or placebo ointment. The placebo ointment is the same as the pharmaceutical compositions of the invention except that the spiro-oxindole compound is not present. Randomization will be stratified by the R1150W underlying genotype in the SCN9A gene: homozygous minor allele (positive, AA), heterozygous (positive, AG), and homozygous common allele (negative, GG).

### Primary Objective

The primary objective of this study is to evaluate the efficacy of 4 weeks of periodic administration of a topical pharmaceutical composition of the invention comprising 4% (w/w) of the spiro-oxindole compound and of a pharmaceutical composition of the invention comprising 8% (w/w) of the spiro-oxindole compound compared with placebo for the relief of pain of primary osteoarthritis of the target knee as assessed by the change from baseline (the 5 days prior to randomization [Days −5 to −1]) to the last 5 days of treatment (Days 24 to 28) in average evening pain intensity upon walking on a flat surface (WOMAC Question 1). Patients are to respond to WOMAC Question 1 based on average pain upon walking since the last assessment or over the past 12 hours.

The WOMAC is a widely-used, validated, patient-reported questionnaire (Bellamy N, Buchanan W W, Goldsmitt C H, Campbell J, Stitt L W, “Validation study of WOMAC: a health status instrument for measuring clinically important patient relevant outcomes to antirheumatic drug therapy in patients with osteoarthritis of the hip or knee”, J Rheumatol 1988; 15:1833-40) that can be used to assess pain, stiffness, and physical function of daily activities in patients with osteoarthritis of the knee. It consists of 24 items separated into 3 domains (pain [5 items], stiffness [2 items], and physical function[17 items]). Pain in the affected joint is assessed when walking on a flat surface, going up/down stairs, at night, sitting/lying and standing upright. Stiffness of the affected joint is assessed after first awakening and after periods of inactivity. Physical function of the affected joint is assessed for daily activities such as descending stairs, ascending stairs, getting out of a chair, remaining in a standing position, bending, walking on a flat surface, getting in and out of a car, shopping, putting socks/stockings on, getting out of bed, taking socks/stockings off, lying in bed, getting in and out of bath, sitting, toileting, heavy domestic duties, and light domestic duties. The visual analog scale (VAS) format is used by the patient in answering the WOMAC Question 1. In this format, the possible score for each item ranges from 0 to 100 mm. The WOMAC pain subscale consists of the 5 items in the pain domain and the possible score ranges from 0 to 500 mm. WOMAC Question 1 is part of the pain domain and asks patients to rate their pain in the target knee while walking on a flat surface.

### Primary Efficacy Endpoint

The primary efficacy endpoint for this study is change from baseline (the 5 days prior to randomization [Days −5 to −1]) to the last 5 days of treatment (Days 24 to 28) in average evening pain intensity in the target knee when walking on a flat surface (WOMAC Question 1).

### Secondary Objectives

The secondary objectives of the study are as follows:
1. To evaluate the efficacy of a topical pharmaceutical composition of the invention (as 4% (w/w) and 8% (w/w) ointments) compared with placebo by examining the following:
   (a) Change from baseline (the 5 days prior to randomization [Days −5 to −1]) to the last 5 days of treatment (Days 24 to 28) in average daily WOMAC pain subscale score for the target knee.
   (b) change from baseline (the 5 days prior to randomization [Days −5 to −1]) to the last 5 days of treatment (Days 24 to 28) in average morning pain intensity upon walking on a flat surface (WOMAC Question 1) for the target knee.
   (c) Change from baseline (randomization visit) to the week 4 visit in the WOMAC physical function subscale score (target knee).
   (d) Change from baseline (randomization visit) to the week 4 visit in the WOMAC stiffness subscale score (target knee).
   (e) 30% and 50% responder rates for average evening pain intensity in the target knee upon walking on a flat surface (WOMAC Question 1) during the last 5 days of treatment (Days 24 to 28) compared with baseline (the 5 days prior to randomization).
   (f) Change from baseline (randomization visit) to the week 4 visit in the Pain Quality Assessment Scale-Revised (PQAS-R). The PQAS-R is a standardized questionnaire that measures various aspects of a patient's pain (Jensen M P, Lin C P, Kupper A E, Galer B S, Gammaitoni A R, “Cognitive testing and revision of the Pain Quality Assessment Scale”, Clin J Pain 2013; 29:400-10). There are 19 questions in the PQAS-R that ask the patient to rate the various aspects (intensity, sharpness, heat, cold, etc.) of his/her pain over the past week on average on a scale of 0 to 10 (0=not [aspect] and 10=the most or worst imaginable [aspect]). In addition, there is a question that asks the patient to select the single description (intermittent, variable, or stable) that best describes his/her pain.
   (g) Patients' global assessment of treatment at the week 2 visit and the week 4 visit as measured by the Patient Global Impression of Change (PGIC) scores. The PGIC is a standardized self-report tool that measures the change in a patient's overall status rating since the start of treatment (Hurst H, Bolton J, “Assessing the clinical significance of change scores recorded on subjective outcome measures”, Journal of Manipulative Physiological Therapeutics (JMPT) 2004; 27:26-35). The 7-point scale is as follows:
      1. very much improved
      2. much improved
      3. minimally improved
      4. no change
      5. minimally worse
      6. much worse
      7. very much worse
   (h) Change from baseline (randomization visit) to the week 2 and week 4 visits in patients' global assessment of treatment as measured by the Patient Global Assessment (PGA) score. The PGA is a simple self-report tool for measuring the overall way osteoarthritis is affecting the patient at a particular point in time (Anderson J K, Zimmerman L, Caplan L, Michaud K, “Measures of rheumatoid arthritis disease activity”, Arthritis Care Res 2011; 63:S14-S36). There is 1 question: “Considering all the ways your arthritis affects you, how are you feeling today?” The response is provided on a VAS from 0 (very poor) to 100 (very good)
   (i) Responder analyses to determine the percentage of patients in each treatment group that are responders based on Outcome Measures in Rheumatoid Arthritis Clinical Trials-Osteoarthritis Research Society International (OMERACT-OARSI) criteria at week 4. The OMERACT-OARSI responder criteria are a method (composite index) for arriving at a simple yes or no answer to whether a patient is a responder to any particular osteoarthritis therapy (Pham T, van der Heijde D, Lassere M, Altman R D, Anderson J J, Bellamy N, et al., “Outcome variables for osteoarthritis clinical trials: the OMERACT-OARSI set of responder criteria”, J Rheumatol 2003; 30:1648-54). It can be used with any validated measures to assess the domains of pain, function, and patient global assessment. In this study, the WOMAC will be used to provide the pain and physical function domains while the PGA will be used to provide the patient global assessment domain. The OMERACT-OARSI responder criteria mandate specific changes (both absolute and relative/proportional) from baseline in particular domains to label a patient as a responder. To be a responder, the patient has to have one of the following at a minimum:
      1. high improvement (at least a 50% improvement from baseline and an absolute change of at least 20 on a scale of 1 to 100) in either the pain or function domain
      2. moderate improvement (at least a 20% improvement from baseline and an absolute change of at least 10 on a scale of 1 to 100) in at least 2 of the 3 domains
2. To estimate the population pharmacokinetic parameters of the spiro-oxindole compound and explore covariate effects on pharmacokinetics:
   (a) Calculating population pharmacokinetic parameters such as clearance (CL/F) and volume of distribution (V/F) following topical administration of the 4% and 8% pharmaceutical compositions.
   (b) Identifying clinically relevant covariates (e.g., age, body weight, CYP3A4 and/or CYP2C19 polymorphisms) affecting the spiro-oxindole compound pharmacokinetics as data permit.
3. To evaluate the safety of the topical 4% and 8% ointment treatment compared with placebo as assessed by the following at specific time points throughout the study based on the schedule of events:
   (a) Occurrence of adverse events throughout the study.
   (b) Clinical safety laboratory (serum chemistry, hematology, and urinalysis) test results.
   (c) Vital signs (heart rate, respiratory rate, body temperature, and blood pressure) measurements.
   (d) ECG findings.
   (e) Physical examination findings.
   (f) Dermal irritation findings
   (g) Concomitant medication usage throughout the study.

### Secondary Efficacy Endpoints

The secondary efficacy endpoints for this study are as follows:
1. Change from baseline (the 5 days prior to randomization [Days −5 to −1]) to the last 5 days of treatment (Days 24 to 28) in average daily WOMAC pain subscale score for the target knee.
2. Change from baseline (the 5 days prior to randomization [Days −5 to −1]) to the last 5 days of treatment (Days 24 to 28) in average morning pain intensity upon walking on a flat surface (WOMAC Question 1) for the target knee.
3. Change from baseline (randomization visit) to the week 4 visit in the WOMAC physical function subscale score (target knee).
4. Change from baseline (randomization visit) to the week 4 visit in the WOMAC stiffness subscale score (target knee).
5. 30% responder rate for average evening pain intensity in the target knee upon walking on a flat surface (WOMAC Question 1) during the last 5 days of treatment (Days 24 to 28) compared with baseline (the 5 days prior to randomization).
6. 50% responder rate for average evening pain intensity in the target knee upon walking on a flat surface (WOMAC Question 1) during the last 5 days of treatment (Days 24 to 28) compared with baseline (the 5 days prior to randomization).
7. Change from baseline (randomization visit) to the week 4 visit in the PQAS-R score.
8. PGIC scores after 2 weeks and 4 weeks of study drug treatment.
9. Change from baseline (randomization visit) to the week 2 and week 4 visits in the PGA score.
10. Percentage of patients who are responders per OMERACT-OARSI criteria at week 4.

### Exploratory Objectives

The exploratory objectives of the study are to evaluate the topical 4% and 8% ointment compared with placebo by examining the following:
1. Change from baseline to week 1 (5-day baseline to the last 5 days of treatment [Days 3 to 7] of week 1) in average evening pain intensity upon walking

(WOMAC Question 1) for the target knee
2. Change from baseline to week 1 (5-day baseline to the last 5 days of treatment [Days 3 to 7] of week 1) in average daily WOMAC pain subscale score for the target knee
3. Change from baseline to week 2 (5-day baseline to the 5-day period before the week 2 [Day 15] visit) in average evening pain intensity upon walking (WOMAC Question 1) for the target knee
4. Change from baseline to week 2 (5-day baseline to the 5-day period before the week 2 [Day 15] visit) in average daily WOMAC pain subscale score for the target knee
5. Change from baseline (randomization visit) to the week 2 visit in the WOMAC physical function subscale score (target knee)
6. Change from baseline (randomization visit) to the week 2 visit in the WOMAC stiffness subscale score (target knee)
7. Use of rescue medication for pain associated with osteoarthritis of a joint. During the course of the treatment period, the patients will be provided with TYLENOL® (McNeil Consumer Healthcare Division of McNEIL PPC, Inc.; generic name acetaminophen or paracetamol) (500 mg tablets) in bottles of 100 tablets and allowed to take 1 to 2 tablets per dose every 4 to 6 hours as needed up to 4 tablets or 2000 mg per day (over a 24-hour period) for rescue relief of OA pain. Rescue medication will be provided at the screening visit. Rescue medication compliance will be checked at all visits until used rescue medication is collected at the baseline or randomization visits for patients not continuing in the study, at the week 4 visit for patients who complete the treatment period, or at the ET visit for those who prematurely discontinue study drug.
8. Analyses of primary and secondary efficacy endpoints with patients stratified by their R1150W polymorphism status: homozygous minor allele (positive, AA) and heterozygous (positive, AG) versus homozygous common allele (negative, GG). Those patients who carry the R1150W polymorphism have a nucleotide containing adenine (A) instead of guanine (G) in the rs6746030 single nucleotide polymorphism (SNP) in the SCN9A gene. This change causes an arginine to tryptophan amino acid residue change at position 1150 in the alpha subunit of the voltage-gated sodium channel Na_{V}1.7. Those who carry the R1150W polymorphism may be heterozygous (GA) or homozygous (AA) for the minor allele. This R1150W polymorphism in the SCN9A gene may affect pain perception in patients with osteoarthritis (Reimann F, Cox J J, Belfer I, Diatchenko L, Zaykin D V, McHale D P, et al., “Pain perception is altered by a nucleotide polymorphism in SCN9A”, Proc Natl Aced Sci 2010; 107:5148-153).
9. Change from baseline (randomization visit) to the week 4 visit in urine and blood soluble biomarkers.
10. Analyses of genetic variables may be explored in association with response variables, including efficacy, safety, pharmacokinetics, and/or pain characteristics.

### Exploratory Efficacy Endpoints

The exploratory efficacy endpoints for this study are as follows:
1. Change from baseline to week 1 (the 5 days prior to randomization to the last 5 days of treatment [Days 3 to 7] of week 1) in average evening pain intensity upon walking (WOMAC Question 1) for the target knee.
2. Change from baseline to week 1 (the 5 days prior to randomization to the last 5 days of treatment [Days 3 to 7] of week 1) in average daily WOMAC pain subscale score for the target knee.
3. Change from baseline to week 2 (the 5 days prior to randomization to the 5 days of treatment before the week 2 [Day 15] visit) in average evening pain intensity upon walking (WOMAC Question 1) for the target knee.
4. Change from baseline to week 2 (the 5 days prior to randomization to the 5 days of treatment before the week 2 [Day 15] visit) in average daily WOMAC pain subscale score for the target knee.
5. Change from baseline (randomization visit) to the week 2 visit in the WOMAC physical function subscale score (target knee).
6. Change from baseline (randomization visit) to the week 2 visit in the WOMAC stiffness subscale score (target knee).
7. Rescue (for osteoarthritis pain) medication usage.
8. Analyses of primary and secondary efficacy endpoints with patients stratified by their R1150W polymorphism status: homozygous minor allele (positive, AA) and heterozygous (positive, AG) versus homozygous common allele (negative, GG).
9. Change from baseline (randomization visit) to the week 4 visit in urine and blood soluble biomarkers
10. Analyses of genetic variables may be explored in association with response variables, including efficacy, safety, pharmacokinetics, and/or pain characteristics

### Safety Variables and Endpoints

The safety of the study drug (4% and 8% ointment) will be assessed throughout the study by evaluating adverse events, clinical safety laboratory test results, vital signs measurements, ECG and physical examination results, and concomitant medication usage.

### Diagnosis and Criteria for Inclusion

Patients may be included in the study if they meet all of the following criteria:
1. Patient is between 40 and 85 years of age, inclusive, with a body mass index (BMI) between 18 and 32 kg/m², inclusive, at the screening visit.
2. Patient has primary osteoarthritis in a single knee (target knee) confirmed by American College of Rheumatology criteria for diagnosis of osteoarthritis and radiograph of target knee rated as 2 to 3 on the Kellgren-Lawrence scale as confirmed by a blinded central study radiologist (digital or digitized radiographs within the 6 months prior to screening acceptable).
3. Symptomatic, primary osteoarthritis of the knee with pain is present for most days in the previous 3 months before the screening visit.
4. For patients taking non-steroidal anti-inflammatory drugs (NSAIDs) or other analgesics at the time of the screening visit, the following criteria must be met:
   (a) Patient has visual analog scale (VAS) scores mm and <90 mm when rating WOMAC Question 1 (pain on walking) in the target knee at the screening visit.
   (b) Patient demonstrates a ‘flare’ of pain on walking in the target knee at the baseline visit following a washout of the regular prior osteoarthritis therapy defined as baseline visit WOMAC Question 1 score ≧50 mm and <90 mm and worsening by at least 5 mm compared with the score at the screening visit.
5. For patients not taking analgesics at the time of the screening visit, VAS score must be ≧50 mm and <90 mm when rating WOMAC Question 1 in the target knee at the screening and baseline visits.
6. Patient has an average VAS score of ≧50 mm when rating WOMAC Question 1 in the evening during the 5 days prior to the randomization visit.
7. Patient has a VAS score of <20 mm when rating WOMAC Question 1 (pain on walking) in the contralateral knee or other joints at the screening and randomization visits.
8. During the baseline period, patients must properly report pain scores for at least 4 of the 5 daily morning recordings for WOMAC Question 1 only and at least 4 of the 5 daily evening recordings of WOMAC Question 1 (as part of the full WOMAC pain subscale) during the 5 days immediately before the randomization visit.
9. If female:
   (a) The patient cannot become pregnant because she is surgically sterile (hysterectomy or tubal ligation) or post-menopausal for at least 6 months.
   OR
   (b) If fertile, the patient is not pregnant and has negative pregnancy tests at both the screening and randomization visits, and agrees to use an acceptable method of contraception (including oral contraceptives, hormone implant, intrauterine device, spermicide with barrier method, surgically sterile male sexual partner[s], or no sexual partners) for the duration of the study.
10. If male:
   The patient must agree to use a barrier method of contraception in combination with a spermicide with any female partner unless she cannot become pregnant because she is surgically sterile (hysterectomy or tubal ligation) or post-menopausal for at least 6 months, or she is fertile but using an acceptable method of contraception (i.e., oral contraceptives, hormone implant, or intrauterine device).
11. Patient must sign written informed consent form and be willing to comply with all study procedures and restrictions.
12. Except for osteoarthritis, patient is judged by the investigator to be medically healthy and able to participate in the study.

### Criteria for Exclusion

Patients will be excluded from participating in this study if they meet one or more of the following criteria:
1. Patient has secondary or inflammatory arthritis of the knee such as psoriasis, rheumatoid arthritis (RA), gout, other primary bone disease, or acute trauma.
2. Patient has chondrocalcinosis on radiography if associated with a history of pseudogout or inflammatory flare-ups. Chondrocalcinosis is allowed if it is deemed as an “asymptomatic” radiological finding only.
3. Patient has a VAS score of 100 mm for WOMAC Question 1 on one or more occasions during the baseline period or VAS scores 90 mm on more than two occasions during the baseline period.
4. Patient has a history of fibromyalgia.
5. Patient has any painful or disabling conditions that in the opinion of the investigator may confound assessment of pain scoring.
6. Patient has uncontrolled cardiac, renal, hepatic or other systemic disorders that in the opinion of the investigator may jeopardize the patient.
7. Patient uses Class Ic anti-arrhythmic drugs such as flecainide or propafenone.
8. Patient has a resting heart rate <45 or >100 beats per minute (bpm), QRS ≧120 msec (including complete left and/or right bundle branch block [LBBB or RBBB]), QTcF ≦320 msec or ≧470 msec, and, for patients in sinus rhythm, PR <120 msec or ≧220 msec.
9. Patient has second or third degree atrioventricular block unless treated with a permanent pacemaker.
10. Patient has uncontrolled atrial fibrillation or flutter (ventricular rate >100 bpm).
11. Patient has congenital arrhythmia syndromes (e.g., Brugada syndrome or long or short QT syndrome).
12. Patient has myocardial infarction within the past 12 months or current unstable angina, coronary ischemia, or heart failure.
13. Patient has significant edema or skin disorder (including sores, rashes, or ulcers) at the target knee and surrounding area.
14. Patient has a history of total or partial knee replacement in either leg.
15. Patient had a major reconstructive knee surgery or arthroscopy of the target knee within 6 months before the screening visit.
16. Patient plans knee replacement or other knee surgery during the clinical study.
17. Patient needs daily oral corticosteroids during the clinical study (patients taking oral corticosteroids at screening must be able to washout before randomization). Inhaled corticosteroids for asthma are acceptable.
18. Patient had intra-articular corticosteroid injection into the target knee within 90 days prior to the screening visit or into any other joint within 30 days prior to the screening visit.
19. Patient had intra-articular viscosupplementation (i.e., EUFLEXXA®, Ferring Pharmaceuticals Inc; SYNVISC®, Genzyme Corporation; or similar treatment) in the knees within 3 months prior to the screening visit.
20. Patient is unable or unwilling to discontinue opioid and/or other prescription analgesics for control of osteoarthritis pain.
21. Patient is intolerant to study drug, its excipients, and/or acetaminophen.
22. Patient uses any over-the-counter (OTC) analgesic or arthritic medication/therapy for the duration of the study (from the baseline period until the end of treatment [week 4] visit) except for permitted rescue (for osteoarthritis pain) medications. Stable therapy of >30 days for aspirin (up to 81 mg/day) is allowed as cardiovascular prophylaxis.
23. Patient uses any non-pharmacological pain management techniques (e.g., physical techniques, physiotherapy, massage therapy, bracing, taping, acupuncture, biofeedback, and/or psychological support) unless the patient has been undergoing/receiving the technique for months at the time of the screening visit and maintains the technique as stable therapy for the duration of the study.
24. Patient uses any OTC oral medications such as glucosamine or chondroitin sulfate products, unless the patient has been receiving the medication for months at the time of the screening visit and maintains the medication as stable therapy for the duration of the study.
25. Patient uses any of the following CYP3A4 or CYP2C19 inhibitors or substrates: Alfentanil, Alprazolam, Amiodarone, Amlodipine, Aprepitant, Atorvastatin, Carbamazepine, Cimetidine, Ciprofloxacin, Cisapride, Clarithromycin, Cyclosporine, Dihydroergotamine, Diltiazem, Disopyramide, Ergotamine, Erythromycin, Estrogens (oral contraceptives), Ethosuximide, Etoposide, Felodipine, Fentanyl, Fluconazole, Fluoxetine, Fluvoxamine, Isoniazid, Isradipine, Itraconazole, Ketoconazole, Lidocaine, Lovastatin, Methadone, Metronidazole, Miconazole, Midazolam, Modafinil, Nefazodone, Nicardipine, Nifedipine, Nimodipine, Nisoldipine, Norfloxacin, Omeprazole, Ondansetron, Oral Contraceptives, Phenytoin, Pimozide, Quinidine, Quinine, Rifabutin, Roxithromycin, Sertraline, Sildenafil, Simvastatin, Sirolimus, Tacrolimus, Tamoxifen, Thioridazine, Tiagabine, Tolterodine, Topiramate, Trazodone, Triazolam, Troleandomycin, Verapamil, Voriconazole, Warfarin, Zafirlukast or Zileuton.
26. Patient uses any topical/cosmetic products (i.e., lotions, tanning products, etc.) on the target knee.
27. Patient has a history of alcohol or drug abuse within 1 year prior to the screening visit, or a positive urine drug test at the screening visit for cocaine, marijuana, opioids, amphetamines, methamphetamines, benzodiazepines, barbiturates, methadone, and/or tricyclic antidepressants unless explained by the use of prescription medication.
28. Patient is pregnant or breast-feeding at the time of the screening visit.
29. Patient has findings in laboratory data, vital signs measurements, or upon physical examination at either the screening or baseline/randomization visits that, in the opinion of the investigator, may pose undue risk to the patient or may interfere with study data interpretation.
30. Patient was previously randomly assigned to treatment in this study and received/subsequently discontinued study drug.
31. Patient used another investigational drug within 30 days or 5 half-lives (whichever is longer) prior to the planned first day of study drug application (Day 1) in this study.
32. Patient is a study center or sponsor employee who is directly involved in the study or the relative of such an employee.
33. Any other reason that would make the patient, in the opinion of either the investigator or the sponsor, unsuitable for the study.

### General Design and Methodology

This is Phase 2, multicenter, randomized, double-blind, parallel group, placebo-controlled study to evaluate the safety and efficacy of the 4% pharmaceutical composition of the invention in ointment form (containing 4% (w/w) of the spiro-oxindole compound and the 8% pharmaceutical composition of the invention in ointment form (containing 8% (w/w) of the spiro-oxindole compound compared with placebo ointment applied topically twice a day (bid) to one knee (i.e., the target knee) for 4 weeks (Day 1 through Day 28) in patients with primary osteoarthritis affecting the knee. For each patient, there will be a total of 6 visits to the study center and 1 telephone contact as follows:

1. Visit 1: Screening visit (up to 28 days before randomization/first administration of study drug)

2. Washout phone contact (approximately 1 week after the screening visit)

3. Visit 2: Baseline visit (Day −8±3)

4. Visit 4: Randomization visit (Day 1 [this is the day after Day −1 and the first day of study drug application])

5. Visit 4: Week 2 visit (Day 15±1)

6. Visit 5: Week 4 visit (Day 29)

7. Visit 6: Follow-up visit (Day 57±3)

The screening period consists of the screening visit (informed consent and preliminary eligibility assessment); washout phone contact during which eligibility based on laboratory test results and the central radiologist's interpretation of the target knee radiograph will be reviewed and, as needed, patients will be given instructions to washout (discontinue) current osteoarthritis therapy including NSAIDs; washout period (if needed) of variable/flexible length during which appropriate patients will discontinue osteoarthritis therapy; baseline visit at which eligible patients will be given an electronic diary (eDiary); and the baseline period during which baseline pain assessments will be made. Patients who do not require washout of NSAIDs/other analgesics will have the baseline visit (Day −8) as soon as screening assessments confirm eligibility and the baseline visit can be scheduled. During the baseline period, rescue medications for osteoarthritis pain will not be allowed. Rescue medication will also not be allowed during the last 5 days of treatment (Days 24 to 28). From the baseline visit onwards, patients will use the eDiary to record responses to WOMAC Question 1 by itself each morning (0700±2 hours) and responses to the 5-item WOMAC pain subscale (including WOMAC Question 1) each evening (1900±2 hours), and any rescue medication usage. Also, patients will be instructed to maintain (hold constant) their current level of physical activity from the baseline visit through the end of treatment [week 4] visit. On all visit days, between the eDiary and the visit activities, the equivalent of a full WOMAC (pain, physical function, and stiffness subscales) will be completed for the target knee. At the screening, Day 1, and Day 29 visits, WOMAC Question 1 (pain on walking) will also be recorded for the contralateral knee.

At the randomization visit (Day 1), eligible patients will be randomly assigned via IRT in a 1:1:1 fashion to 1 of 3 treatment groups: 4% ointment, 8% ointment, or placebo ointment. Randomization will be stratified by test results of the pharmacogenomic sample collected at the screening visit (homozygous minor allele [positive, AA], heterozygous [positive, AG], and homozygous common allele [negative, GG]) for the R1150W polymorphism in the SCN9A gene. The patient will be instructed on how to apply study drug. Under study center staff supervision, the patient will apply the first dose of blinded study drug and record the date/time in the eDiary. Baseline efficacy assessments (equivalent of the full WOMAC for the target knee between visit activities and the eDiary, WOMAC Question 1 for the contralateral knee, PGA, and PQAS-R) will be conducted at this visit. For patients in the pharmacokinetic subset (approximately 40 patients per pharmaceutical composition group and 10 patients in the placebo group and selected via IRT at designated clinical study centers), baseline pharmacokinetic samples (blood) will be collected before study drug administration. For all patients, blood and urine samples for biomarker analyses will be taken.

During the 4-week treatment period, patients will apply double-blind study drug to the target knee twice daily, once in the morning (0700±2 hours) and again in the evening (1900±2 hours). The first dose of study drug will be applied at the clinic on Day 1. Regardless of the clock time of the first dose application at the randomization visit, the evening dose for Day 1 should be applied at 1900±2 hours. The last dose of study drug will be applied at home on the evening of Day 28. The morning study drug applications will be done after recording the response for WOMAC Question 1 by itself for the target knee (i.e., the knee having the osteoarthritic pain). The evening drug applications will be done after recording the responses for the 5-item WOMAC pain subscale for the target knee. After the baseline visit when the eDiary is provided, the eDiary will be used to record the WOMAC Question 1 and WOMAC pain subscale responses, osteoarthritis rescue medication use, and the dates/times of study drug administration.

Patients will return to the study center for a week 2 visit at Day 15 (±1) and again for a week 4 visit at Day 29. Routine efficacy (equivalent of the full WOMAC for the target knee via eDiary and visit activities, PGA, PQAS-R, and PGIC) and safety assessments will be done at both visits. For Day 29 only, WOMAC Question 1 will be recorded for the contralateral knee. Pharmacokinetic samples will also be taken at both visits for patients in the pharmacokinetic subset at the following times: hour after the dose at the week 2 visit and at the week 4 (Day 29) visit which will take place the morning after the last dose taken on the evening of Day 28. Also, the dates/times of the pharmacokinetic sample collections will be recorded. Also, at the week 4 visit, the eDiary will be collected along with the tubes of study drug and bottles of rescue medication and blood and urine samples for biomarker analyses.

Four weeks after the week 4 visit, the patients who completed the double-blind treatment period will return to the study center for a follow-up visit. Activities will include efficacy and safety assessments for all patients and pharmacokinetic sampling (including recording of the date/time of sampling) for those in the pharmacokinetic subset.

Patients who prematurely discontinue study drug will have an early termination (ET) visit (same activities as the follow-up visit plus eDiary and unused study drug/rescue medication collection [and compliance checks] as well as collection of blood/urine samples for biomarker analyses) within 2 weeks after the last study drug administration. Any treatment-emergent adverse event or serious adverse event will be followed until resolution or return to baseline; otherwise, this ET visit will be the last study visit for these patients.

### Pharmacokinetic Study

Blood samples will be taken from the approximately 90 patients (40 patients from each pharmaceutical composition group and 10 patients in the placebo group) in the pharmacokinetic subset at the randomization visit (before first study drug application), at the week 2 visit (1 hour after the morning dose), at the week 4 visit (morning after the last dose on the evening of Day 28), and at the follow-up visit. The dates/times of the sample collections will be recorded. Plasma samples will be analyzed for the spiro-oxindole compound. Descriptive statistics for the spiro-oxindole compound plasma concentrations will be included in the clinical study report (CSR). Population pharmacokinetic parameters, such as clearance (CL/F) and volume of distribution (V/F), following topical administration of the pharmaceutical compositions to patients with osteoarthritis will be estimated. Clinically relevant covariates affecting the pharmacokinetic of the spiro-oxindole compound will be identified as data permits. These results will be reported in a population pharmacokinetic report separate from the CSR.

### Pharmacodynamic Analyses

Blood and urine samples for assessment of plasma soluble biomarkers and urinary C-terminal telopeptide of type II collagen (CTXII) will be collected from all patients at the randomization visit (before the first dose) and at the week 4 or ET visit.

The samples will be retained for a maximum of 15 years after the last patient last visit and may be analyzed for biomarkers using proteomics, metabolomics, and/or other methodologies during this period.

### Pharmacogenomic Analyses:

Two blood samples for pharmacogenomic analyses will be taken from all patients at the screening visit. Patients who refuse to give these blood samples will be excluded from the study. One sample will be analyzed to identify the nucleotide (G or A) underlying the R1150W polymorphism in the SCN9A gene and, perhaps, whether there are any other sequence variants in the SCN9A gene region. All samples will be retained for a maximum of 15 years after completion of the study and may be analyzed for other genetic variations potentially associated with pain signaling or drug response including efficacy, metabolism, and safety parameters. For subjects in the pharmacokinetic subset, a pharmacogenomic blood sample may be used to assess polymorphisms of the CYP3A4 and CYP2C19 genes. Depending on the distribution of allelic variations for CYP3A4 and CYP2C19, these results may be incorporated as covariates in the current or future population pharmacokinetic analyses. Pharmacogenomic results for the R1150W polymorphism will be included in the CSR as part of the analyses of primary and secondary efficacy endpoints stratified by R1150W polymorphism status: homozygous minor allele (positive, AA) and heterozygous (positive, AG) versus homozygous common allele (negative, GG).

### Statistical Considerations:

Patient demographic and baseline characteristics, including medical history, prior medications, and ECG findings will be examined to assess the comparability of the treatment groups and will be summarized using descriptive statistics. For continuous variables, descriptive statistics (number [n], mean, standard deviation, standard error, median, minimum, and maximum) will be provided. For categorical variables, patient counts and percentages will be provided. Treatment groups will be compared for all continuous variables, using an analysis of variance (ANOVA) with treatment group and study center as factors. Treatment groups will be compared for all categorical variables using a Pearson's chi square (or Fisher's exact test if cell sizes are too small).

The full analysis set (FAS) of all patients in the intent-to-treat (ITT) population (all patients randomly assigned to treatment) who receive at least 1 dose of study drug and have at least 1 post-baseline efficacy assessment will be used for all efficacy analyses. Summaries will be presented by treatment group.

The primary efficacy variable (change from 5-day baseline to the last 5 days of treatment in the average evening WOMAC Question 1) will be analyzed using a Mixed Model Repeated Measures (MMRM) model (SAS® MIXED procedure with REPEATED sub-command). The model will include the following fixed effects: categorical week in the study by treatment interaction, study center, and baseline average evening WOMAC Question 1 score. The unstructured covariance matrix for repeated observations within patients will be used. In case the model does not converge, the maximum-likelihood (ML) estimation method will be used instead of the default restricted ML (REML) method. If the model still does not converge, then simpler covariance structures with less parameters will be used in the following order until the model does converge: heterogeneous autoregressive (1) (ARH(1)); heterogeneous compound symmetry (CSH); autoregressive (1) (AR(1)); and compound symmetry (CS). The estimated week 4 mean change from baseline in average evening WOMAC Question 1 score will be compared between the active groups and the placebo group.

The secondary efficacy variable, change from 5-day baseline to the 5-day period before the week 4 [Day 29] visit in average daily 5-item WOMAC pain subscale score for the target knee, will be analyzed in the same way as the primary efficacy variable. Other continuous secondary efficacy endpoints based on WOMAC and the PQAS-R will be analyzed using similar methods.

PGIC scores will be analyzed using a MMRM model with week, treatment, and treatment by week interaction as the fixed factors, and patient as a random factor. The unstructured covariance matrix for repeated observations within patients will be used.

The week 4 responder rate per OMERACT-OARSI criteria will be analyzed using a generalized estimating equation (GEE).

Continuous exploratory efficacy endpoints based on WOMAC will be analyzed in the same way as the primary and secondary efficacy endpoints based on WOMAC.

The primary and secondary efficacy analyses will be repeated with patients stratified by R1150W polymorphism status: homozygous minor allele (positive, AA) and heterozygous (positive, AG) versus homozygous common allele (negative, GG).

The use of rescue medications for pain associated with osteoarthritis in a joint by patients in each treatment group will be compared.

No other rescue medications will be provided or allowed from the washout phone contact through the week 4 or ET visit. Patients will not be permitted to use rescue medication during the baseline period (days −8 to −1) and during the final week of treatment (the 5-day period before the week 4 [day 29] visit). During the washout period, rescue medication use will be collected as a concomitant medication. During the treatment period, rescue medication use will be recorded using the eDiary.

All adverse events will be coded using the Medical Dictionary for Regulatory Activities (MedDRA). Each patient will be counted only once in each preferred term or system organ class (SOC) category for the analyses of safety. Summaries will be presented for all adverse events (overall and by severity), adverse events determined by the investigator to be related to study treatment (i.e., reasonable possibility; defined as related or with missing relationship) (overall and by severity), serious adverse events, and adverse events causing withdrawal from study drug treatment and/or the study. Summaries will be presented by treatment group and for all patients. Patient listings of serious adverse events and adverse events leading to withdrawal will be presented.

Changes in laboratory and vital signs measurement data will be summarized descriptively. All values will be compared with pre-specified boundaries to identify potentially clinically significant changes or values, and such values will be listed.

The use of concomitant medications will be summarized by therapeutic class using descriptive statistics. Concomitant medications will include all medications taken while the patient is treated with study drug.

For continuous variables, descriptive statistics (n, mean, standard deviation, standard error, median, minimum, and maximum) will be provided for actual values and changes from baseline to each time point. For categorical variables, patient counts and percentages will be provided. Descriptive summaries of serious adverse events, patient withdrawals due to adverse events, and potentially clinically significant abnormal values (clinical laboratory or vital signs) based on predefined criteria will also be provided.

If any patient dies during the study, a listing of deaths will be provided and all relevant information will be discussed in the patient narrative included in the CSR.

The population pharmacokinetic analyses will be detailed in a population pharmacokinetic analysis plan for this study. Results will be reported separately from the CSR. However, descriptive statistics for plasma concentration of the spiro-oxindole compound will be included in the CSR.

### Sample Size Considerations

It is estimated that 100 completers (patients who finish the double-blind treatment period) per treatment group will provide 80% power with a 2-sided test at a 5% significance level to detect a treatment effect of 0.8 cm (8 mm on the 100 mm VAS) in the change from baseline (5-day baseline) to week 4 (the 5-day period before the week 4 visit), with a standard deviation (SD) of 2.0 cm, in the average evening WOMAC Question 1, pain upon walking on flat surface, score.

In order to ensure 100 completers per treatment group, approximately 125 patients will be randomly assigned to each treatment group.

### Expected Results

The results of this study are expected to show that the methods of the invention are effective in treating pain associated with osteoarthritis in a joint in a human with minimal or negligible systemic exposure as evidenced by low concentrations of the active ingredient, i.e., the spiro-oxindole compound of the invention, in the plasma of the human during and after treatment.

### Biological Example 4

### A Randomized, Double-Blind, Placebo Controlled, Parallel-Group, Dose Escalation Study to Characterize the Pharmacokinetics and Safety of a Topical Formulation of the Invention

This study is designed to evaluate the systemic exposure of a spiro-oxindole compound under theoretical maximal use conditions. It is anticipated that this study will provide information on the dose-exposure relationship, the single- and multiple-dose pharmacokinetics (PK) of the spiro-oxindole compounds, the relationship between skin and systemic concentrations of the spiro-oxindole compound, and the washout of the spiro-oxindole compound from both the systemic circulation and the dermal compartment. Depending on local tolerability, the study would also provide information to help design additional topical studies.

In general, the maximal concentrations of the spiro-oxindole compound following topical application are determined under “maximal use conditions,” taking into consideration such factors as ointment strength, application volume, application surface area, and frequency and duration of dosing. In two previous Phase 2a studies, the 8% test pharmaceutical composition of the invention was applied twice daily to patients with postherpetic neuralgia (PHN) and patients with erythromelalgia (EM). In the PHN study, the application volume was 7.5 mg/cm² (i.e., 7.5 μL/cm²) and the application surface area ranged from 100 to 400 cm², resulting in a wide range of plasma Cₘₐₓ values which collectively averaged approximately 2 ng/mL. In contrast, with a decrease in application volume (4 mg/cm²) but a much larger application surface area (800-1400 cm²), the average Cₘₐₓ value in patients with EM was 2.9±2.5 ng/mL. While these Cₘₐₓ values are >100-fold lower than the average Cₘₐₓ value observed following multiple oral administrations of 400 mg of the spiro-oxindole compound, the highest observed individual concentration following any topical administration to date is 14.5 ng/mL.

The results of the previous topical studies suggested that the average steady-state Cₘₐₓ value for the spiro-oxindole compound in plasma following twice daily application of 4 mg ointment/cm² to approximately 7% of body surface area (BSA) would be conservatively estimated at approximately 3 ng/mL, with a maximum observed individual plasma concentration of 14.5 ng/mL. Therefore, assuming linear pharmacokinetic behavior (i.e., systemic exposure is correlated with the amount of ointment applied/surface area), application of 3 mg ointment/cm² to 7% BSA in one group, to 21% BSA in a second group and 53% BSA to a third group in healthy subjects is conservatively estimated to result in average maximal exposures (Cₘₐₓ) in plasma of between 2 to 3 ng/mL, 7 to 9 ng/mL, and 16 to 22 ng/mL, respectively. Nevertheless, the exposure level anticipated in this study is expected to be higher than that observed in any single patient population administered a therapeutic dose a topical pharmaceutical composition of the invention. This study is therefore intended to help in the selection of a topical dose and regimen of a topical pharmaceutical composition of the invention.

Taking into consideration, that patients would apply the topical pharmaceutical compositions of the invention differently for different indications, that patients might have multiple conditions for which the topical pharmaceutical compositions of the invention might be indicated, or that some patients could either misuse or abuse the topical pharmaceutical compositions of the invention by not following directions, the present study is designed to evaluate the systemic exposure of the topical pharmaceutical compositions of the invention under theoretical maximal use conditions. It is anticipated that this study will provide information on the dose-exposure relationship, the single- and multiple-dose pharmacokinetics (PK) of the spiro-oxindole compound, the relationship between skin concentration (in calf tissue) and systemic concentrations of the spiro-oxindole compound, and the elimination of the spiro-oxindole compound from both the systemic circulation and the skin. The study would provide information to help design additional topical clinical pharmacology studies that are required for registration purposes.

### Study Objectives

Accordingly, the primary objective of this study is to characterize the pharmacokinetics (PK) of the spiro-oxindole compound in plasma following single and multiple-dose topical application of a topical pharmaceutical composition of the invention, specifically the 8% test pharmaceutical composition of the invention. The secondary objectives of the study are as follows:
1. To evaluate the single and multiple-dose safety and tolerability of the 8% test pharmaceutical composition of the invention in ointment form.
2. To evaluate the concentrations of the spiro-oxindole compound in skin at various times throughout the study.
3. To qualitatively and/or quantitatively evaluate the concentrations of the spiro-oxindole and metabolites of interest in urine under multiple-dose conditions.
4. To assess intra-epidermal nerve fiber density in skin under multiple-dose conditions compared to baseline.

### Subject Inclusion Criteria

Subjects may be included in this study only if they meet all of the following criteria:
1. Healthy male and/or female subjects aged 18 to 50 years, inclusive at the time of screening.
2. Body Mass Index (BMI) 18.0 and 32.0 kg/m².
3. Able and willing to provide written informed consent.
4. Able and willing to comply with all study procedures and restrictions.

### Subject Exclusion Criteria

Subjects will be excluded from participating in this study if they meet any of the following criteria:
1. History or evidence of clinically significant illness or surgery within 4 weeks prior to Day 1.
2. History or evidence of any clinically significant disease including neurological, dermatological, cardiovascular, endocrine, pulmonary, gastrological, renal, hepatobiliary, hematological, immunological, urologic, psychiatric, metabolic or other major disease. Subjects with Gilbert's syndrome may be included.
3. Presence of open wounds, sunburn, tattoo, major scarring, non-intact or damaged skin (e.g., resulting from exudative dermatitis, eczema, acne, infected lesion, burns) in the proposed application area, presence of significant pain, neuropathy, venous stasis or any other condition in the proposed application area that, in the opinion of the investigator, would interfere with the application of the study drug treatments or biopsies.
4. Missing or amputated limb (e.g., foot, hand, arm, leg).
5. Any clinically significant abnormality (including clinically significant laboratory test result) found at screening or Day −1.
6. Any positive test for hepatitis B surface antigen, hepatitis C antibodies, or antibodies for human immunodeficiency virus (HIV) 1 or 2.
7. Vital sign abnormalities on screening and/or Day −1 including: having a systolic blood pressure <90 or ≧140 mmHg; diastolic blood pressure <50 or ≧90 mmHg; or resting heart rate <40 or >100 beats per minute.
8. ECG abnormalities at screening including: QRS ≧110 msec (including complete left and/or right bundle branch block (LBBB or RBBB)), QTcF ≧320 msec or ≧450 msec and, for subjects in sinus rhythm, PR <120 msec or ≧220 msec. The ECG may be repeated twice at the investigators discretion if initial values are out of range, in which case the mean of the 3 replicates will be used by the investigator to determine whether the subject is eligible for inclusion.
9. Any cardiac abnormality including but not limited to uncontrolled atrial fibrillation or flutter (ventricular rate >100 bpm), second or third degree heart/atrioventricular block, use of a cardiac pacemaker, or any congenital arrhythmia syndrome (e.g., Brugada syndrome, Wolff-Parkinson-White (WPW) syndrome or long or short QT syndrome), myocardial infarction within the past 12 months, or current unstable angina, coronary ischemia or heart failure.
10. History of significant drug or alcohol abuse (as assessed by the DSM IV-TR criteria) within the past year prior to screening.
11. Positive urine drug screen for drugs of abuse and cotinine at screening and/or Day −1.
12. History of significant alcohol consumption within 6 months prior to screening, defined as exceeding 14 units of alcohol per week, where 1 unit=12 fluid ounces of beer, 5 fluid ounces of wine, or 1.5 fluid ounces of hard liquor.
13. Alcohol consumption or positive alcohol test on admission to the clinic on Day −1.
14. Subjects who smoke, have smoked in the last 45 days, or are planning to start smoking during the study, tobacco users, subjects currently using nicotine products (patches, gums, etc.) or subjects with a confirmed positive urine cotinine test at screening and/or Day −1.
15. Use of prescription drugs within 30 days or 5 half-lives (whichever is longer) prior to Day 1.
16. Use of oral over-the-counter (OTC) products (including oral natural health products, vitamins, herbal supplements) within 7 days prior to Day 1 with the exception of intermittent paracetamol up to 2 g/day or ibuprofen up to 800 mg/day.
17. Use of topical application of an OTC medicated or prescription medication or other skin creams/ointments (e.g., moisturizers, balms) in the areas intended for study drug administration within 7 days prior to Day 1.
18. History of severe allergic or hypersensitivity reactions (e.g., angioedema) to any drug including lidocaine or sodium-lauryl-sulfate or any topical OTC cream, balm, or ointment.
19. Use of any investigational drug or participation in any investigational drug or device study within 30 days of screening or 5 elimination half-lives, whichever is longer.
20. Pregnant or nursing females (including positive pregnancy test at screening or Day −1).
21. Females using hormonal contraceptives.
22. Females of childbearing potential who are unable or unwilling to undertake adequate measures to prevent a pregnancy from at least 30 days prior to the first dose of study medication through until 28 days after the last dose of study medication. Adequate measures, include any non-hormonal, medically acceptable method of contraception by subject and/or partner (i.e., abstinence, vasectomy, diaphragm and spermicide, non-hormonal intrauterine device (IUD), condom and vaginal spermicide, and/or surgical sterilization). Females will be considered to not be of child-bearing potential if they are postmenopausal (defined as amenorrheic for at least 1 year AND have serum follicle stimulating hormone (FSH) level of at least 35 IU/L) or have undergone a hysterectomy or bilateral oophorectomy.
23. Males unable or unwilling to undertake adequate measures to prevent a pregnancy from the first dose of study medication through until 28 days after the last dose of study medication (i.e., not using any medically acceptable method of contraception by subject and/or partner which would include abstinence, vasectomy, hormonal birth control, diaphragm and spermicide, IUD, condom and vaginal spermicide, surgical sterilization, and/or postmenopausal partner).
24. Donation or loss of 500 mL or more whole blood within 60 days prior to Day 1 or plasma, white blood cells or platelets within 14 days prior to Day 1, or plans to donate blood during or within 90 days after the study.
25. Shaving or waxing the planned study treatment application area within 7 days prior Day 1.
26. Laser hair removal of the planned study treatment application area within 2 months prior to Day 1.
27. Overabundance of hair on the back and/or legs that, in the opinion of the investigator, would preclude the effective application of study drug to the application area(s).

### Mode of Administration, Administration Rate, and Estimated Dose

The study composition is the 8% test pharmaceutical composition of the invention (the 8% test composition) as defined above in ointment form or a placebo composition (which will have the same composition as the 8% test composition of the invention except that the spiro-oxindole compound is not present and the amount of PEG400 in the placebo composition is increased by the amount of the spiro-oxindole compound if it were present) in ointment form for topical administration. The study compositions, both active and placebo, will be provided to the subjects in 50 g tubes.

All study compositions will be administered topically beginning on the morning of Day 1, continuing every 12 hours (bid) until the morning of Day 8 (i.e., a total of 15 doses over 7.5 days). Subjects in Group 1 will have study composition administered to 7% of body surface area (BSA) (feet only), subjects in Group 2 will have study composition applied to 21% of BSA (feet+lower legs), and subjects in Group 3 will have study composition applied to 53% of BSA (feet+lower legs+thighs+back).

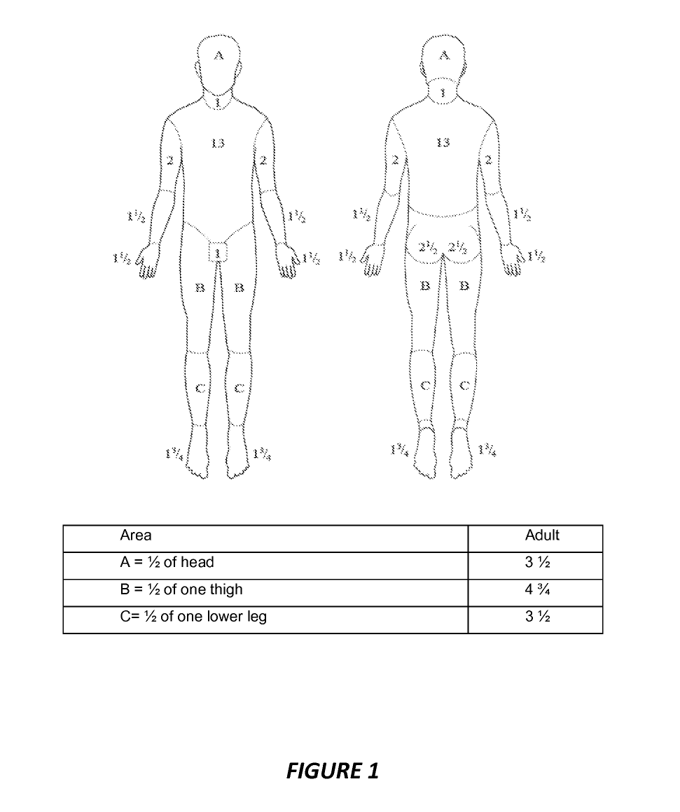

BSA is estimated using the Lund and Browder method (Lund, C. C. and Browder, N. C., Surg Gynaecol Obstet. (1944); 79:352-8) as shown in FIG. 1.

Details of the application areas for the study compositions are as follows:

Applying a study composition to feet: apply a thin layer (3 mg/cm²) of composition to top and bottom of both feet (including toes), covering an area just up to the ankle joint.

Applying a study composition to lower legs (calf areas): apply a thin layer (3 mg/cm²) of composition to entire calf and lower leg areas (i.e., front-to-back and side-to-side) from just below the ankle joint to the mid-line of the knee cap, i.e., where the tibia (lower leg) and femur (thigh) meet (upper limit of Area C on Lund and Browder diagram).

Applying a study composition to upper legs (thigh areas): apply a thin layer (3 mg/cm²) of composition to entire thigh areas (i.e., front-to-back and side-to-side) from the mid-line of the knee cap to as high as the underwear line in the groin, i.e., closest to back view of Lund and Browder chart (upper limit of Area B). Subjects should therefore wear classic briefs/panties (e.g., not boxer shorts, thongs, etc.) and buttocks should be covered.

Applying study composition to the back: apply a thin layer (3 mg/cm²) of composition to entire back area from the middle of the shoulders (i.e., posterior to the mid axillary line), along a theoretical tee-shirt line around the neck, extending downward on the trunk along each arm length to an area at the top of the underwear line at the waist, i.e., see back view of Lund and Browder chart. For women, bras should be removed during application and kept off for at least 30 minutes. Subsequently, the bra should be kept as loose as possible.

The estimated dose or total amount of the spiro-oxindole compound applied to each subject will depend on that individual's overall body surface area (BSA) as shown below, where BSA will be estimated in m² when height (HT) and weight (WT) are recorded in centimeters and kilograms, respectively:

BSA=\sqrt{(HT·WT)/3600}

Prior to each administration of a study composition, a member of the clinic staff will use water to gently clean all the treatment application areas. The treatment/application areas will be gently wiped and lightly patted dry with a soft towel. The treatment areas should be completely dry before application of the composition. All study compositions will be applied by the clinic staff wearing protective (unpowdered) medical gloves. In general, each study composition administration will involve applying the study composition at a dose of 3 mg of composition (approximately equivalent to 3 μL) per cm² of skin. The ointment should be dabbed onto the skin surface and then spread laterally in a thin uniform layer that covers the entire application area. The ointment should be gently massaged (but not rubbed) into the application area.

### Study Design

This study is a single center, randomized, double blind, placebo-controlled, escalating multiple-dose study to investigate the pharmacokinetics and safety of a 8% test pharmaceutical composition of the invention (test composition) administered topically to healthy adult male and female subjects. For each subject, the study duration will be up to 10 weeks, consisting of a screening period of up to 4 weeks, an 11-day inpatient phase (including 7.5 days of dosing) and an outpatient phase (including follow-up visit) of 29 days. There will be a total of 7 visits to the study center as follows:

Visit 1: Screening visit (up to 28 days before the first dose of the study composition.

Visit 2: Inpatient phase (Day −1 to Day 10)

Visit 3: Day 12 visit

Visit 4: Day 15 visit

Visit 5: Day 22 visit

Visit 6: Day 29 visit

Visit 7: Follow-up visit (Day 39±2)

Subjects meeting the study inclusion/exclusion requirements will be randomized 4:1 to receive twice daily ((BID) topical treatment with study composition (i.e., either 8% test pharmaceutical composition in ointment form or placebo composition in ointment form) for 7.5 days (total 15 applications) as shown in Table 7 below:

TABLE 7
TREATMENT SCHEME BY GROUP

| | | | Treatment Area | | Total dose of | Dosing |
|---|---|---|---|---|---|---|
| | Number | Study | | Surface Area | ointment per | Frequency |
| | of | composition | Percent | men/women^{b} | application | and |
| Group | subjects | (ointment) | BSA^{a} | (cm²) | men/women ^{b} | Duration |
| 1 | 12 | 8% test composition | 7%^{c} | ~1420/1210 | ~4.26/3.63 g | Twice daily (BID) |
| | 3 | placebo | | | | for 7.5 days |
| 2 | 12 | 8% test composition | 21%^{d} | ~4260/3630 | ~12.79/10.90 g | Twice daily (BID) |
| | 3 | placebo | | | | for 7.5 days |
| 3 | 12 | 8% test composition | 53%^{e} | ~10760/9170 | ~32.28/27.51 g | Twice daily (BID) |
| | 3 | placebo | | | | for 7.5 days |

^{a}Estimated based on the Lund and Browder method
^{b} Estimated based on an average BSA of 20,300 cm² in men (2.03 m²) and 17,300 cm² in women (1.73 m²). Individual estimates of BSA will vary.
^{c}Both feet (2 × 3.5%) for a total of 7% of BSA.
^{d}Both feet (2 × 3.5%) and both lower legs (2 × 7%) for a total of 21% of BSA.
^{e}Both feet (2 × 3.5%) both lower legs (2 × 7%), both thighs (2 × 9.5%) and back (13%) for a total of 53% of BSA.

Groups will be enrolled and dosed sequentially, with subjects in having the lowest treatment area completing the 11-day inpatient period and blinded evaluation of the safety data before the next group starts treatment at a increased treatment area. Subjects may only enter one group. Any subject who qualifies for randomization in this study but who is not randomized due to practical or scheduling reasons (e.g., the group is already full or due to personal/technical reasons such as family or work-related matters, transportation difficulties or emergencies), may be rolled over to be randomized in a subsequent group provided they are still within the required screening window. In the event that the screening window has elapsed, the subject may be rescreened for the study and will be given a new screening number. Subjects may not be rescreened because they originally failed to meet any of the entry criteria, however prior to disqualification of a subject from study participation and at the discretion of the Investigator, the screening or Day −1 assessments may be repeated in order to determine whether or not a result is sustained or reproducible

During the dosing period each subject enrolled in the study will be admitted to the Clinical Research Unit (clinic) on Day −1. On Day 1 the subject will receive the test or placebo composition and undergo serial assessments for pharmacokinetic and safety monitoring as outlined in Table 8 below. Each subject will be confined to the clinic for a period of approximately 11 days (10 nights), which will include administration of the test or placebo composition on 8 days (total of 15 doses administered) and 3 additional days of pharmacokinetic sampling and safety monitoring before discharge on Day 10. Additional pharmacokinetic sampling and safety assessments will be done on an outpatient basis on Days 12, 15, 22 and 29 and at the Follow-up visit (Day 39±2). Final safety assessments will also be performed at the Follow-up visit.

Skin biopsies will be collected from subjects in Groups 2 and 3 only. These biopsies are being taken for two different purposes. In Group 2, the biopsies will be used for intra-epidermal nerve fiber (IENF) density assessments, whereas those collected in Group 3 will be used for pharmacokinetic purposes.

### Primary and Secondary Measures and Endpoints

A. Primary Pharmacokinetic Measures and Endpoints

Blood samples for the spiro-oxindole compound pharmacokinetic assessments will be collected from all subjects at intervals from Day 1 through to the Follow-up visit. All samples will be assayed for the spiro-oxindole plasma concentrations. Pharmacokinetic variables will be determined as described below.

B. Secondary Pharmacokinetic Measures and Endpoints

Urine samples will be collected from subjects in Cohort 3 only on Days 1 and 8 to determine the concentration of the spiro-oxindole compound in urine. Skin biopsies will be collected from subjects in Group 3 only for assessment of the spiro-oxindole concentrations at intervals from Day 1 to Day 29. Pharmacokinetic variables will be determined, where possible, as described below.

Blood samples for metabolite identification in plasma will be collected from all subjects on Day 1 and at intervals up to 48 hours after the last dose on Day 8.

All urine and skin samples from Group 3 will also be used for exploratory metabolite identification (and quantitation if possible).

Pharmacokinetic samples obtained in this study may also be used for assessment of drug binding to proteins, development or validation of bioanalytical methods and stability, measurement of metabolites, or other purposes related to the absorption, distribution, metabolism, and excretion properties of the investigational product.

### Study Procedures and Assessments

Study procedures and their assessments with their timing are summarized in Table 8 below. Where timings of assessments coincide on Days 1 to 8, the pharmacokinetic blood sample should be collected at the target time. Otherwise, the order of assessments should be as follows: ECG, vital signs, pharmacokinetic blood sample (i.e., at the target time), pharmacokinetic urine sample, and skin biopsy. Exceptions are for the pre-morning dose urine sample and skin biopsies on Day 1 which may be collected in advance of the other assessment.

TABLE 8
STUDY PROCEDURES AND ASSESSMENTS

| | Screening | Study Days | | | | | | | | | | | | | | | |
|---|---|---|---|---|---|---|---|---|---|---|---|---|---|---|---|---|---|
| Procedures and Assessments^{a} | (within 28 days | Inpatient | | | | | | | | | | | Outpatient | | | | Follow-up |
| Study Day | of first dose) | −1 | 1 | 2 | 3 | 4 | 5 | 6 | 7 | 8 | 9 | 10 | 12 | 15 | 22 | 29 | 39 ± 2 |
| Visit | 1 | 2 | | | | | | | | | | | 3 | 4 | 5 | 6 | 7 |
| Informed consent | X | | | | | | | | | | | | | | | | |
| Review eligibility criteria | X | X | | | | | | | | | | | | | | | |
| Medical history | X | | | | | | | | | | | | | | | | |
| Prior and concomitant medication | X | X | X | X | X | X | X | X | X | X | X | X | X | X | X | X | X |
| Physical exam^{b} | X | X | | | | | | | | | | | | | | | X |
| 12-Lead ECG^{c} | X | X | | | X | | | X | | X | | X | X | X | X | X | X |
| Laboratory tests | X | | | | X | | | | | X | | X | | | | | X |
| Follicle Stimulating Hormone (FSH)^{d} | X | | | | | | | | | | | | | | | | |
| Serology tests^{e} | X | | | | | | | | | | | | | | | | |
| Vital signs^{f} | X | X | X | X | X | X | X | X | X | X | X | X | X | X | X | X | X |
| Urine drug screen/cotinine test | X | X | | | | | | | | | | | | | | | |
| Alcohol test | | X | | | | | | | | | | | | | | | |
| Serum pregnancy test^{g} | X | X | | | | | X | | | | | | | | | | X |
| Pharmacogenetic sample | | | X | | | | | | | | | | | | | | |
| Admission to Clinic | | X | | | | | | | | | | | | | | | |
| Dosing of study drug^{h} | | | X | X | X | X | X | X | X | X | | | | | | | |
| Plasma sample for PKⁱ | | | X | X | X | X | X | X | X | X | X | X | X | X | X | X | X |
| Plasma sample for metabolite ID^{j} | | | X | | | | | | | X | X | X | | | | | |
| Skin biopsy ^{k} | | | X | | | | | | | X | | | | X | X | X | |
| Urine collection for PK and metabolite ID^{l} | | | X | | | | | | | X | | | | | | | |
| Bathing^{m} | | | X | X | X | X | X | X | X | X | | | | | | | |
| AE assessment | X | X | X | X | X | X | X | X | X | X | X | X | X | X | X | X | X |
| Dermal irritation | | | X | X | X | X | X | X | X | X | X | X | X | X | X | X | X |
| Discharge from Clinic | | | | | | | | | | | | Xⁿ | | | | | |

^{a}On Days 1 to 8, all assessments will be conducted prior to the morning dose unless otherwise specified.
^{b}Complete physical examination at screening (including recording of height, weight, BMI and BSA) and symptom-based examination on Day −1 and follow-up.
^{c}12-Lead ECGs to be collected at screening (single measurement), on Day −1 (in triplicate), Days 3 and 6 at time 0 (pre-morning dose, single measurements), Day 8 at time 0 (pre-morning dose), 1, 2, 4 and 6 hours post-morning dose (all triplicate measurements), Day 10 (prior to discharge, single measurement) and on Days 12, 15, 22, 29 and Follow-up (all single measurements).
^{d}For postmenopausal females only
^{e}To include Hepatitis B surface antigen, Hepatitis C antibody, and Human Immunodeficiency Virus (HIV) antibody.
^{f}Vital signs to include supine blood pressure, pulse rate, and oral body temperature.
^{g}Females only.
^{h}Study drug will be administered topically every 12 hours (morning and evening) on Days 1 to 7 and in the morning of Day 8.
ⁱAll subjects in Cohorts 1, 2 and 3 will have a PK plasma sample taken at time 0 (pre-morning dose), 1, 2, 3, 4, 6, 8, 10 and 12 hours post-morning dose on Day 1, pre-morning dose (time 0) on Days 2, 3, 4, 5, 6 and 7, and at time 0, 1, 2, 3, 4, 6, 8, 10, 12, 16, 24, 30, 36 and 48 hours as an inpatient, and at 96 hour (Day 12), 168 hours (Day 15), 336 hours (Day 22), 504 hours (Day 29) and 744 ± 48 hours (Day 39 ± 2) following the last dose of study drug on the morning of Day 8.
^{j}Plasma sample to be taken for metabolite identification on Day 1 at time 0 (pre-morning dose) and on Day 8 at 1, 2, 6, 12, 24 and 48 hours after the last dose.
^{k} All subjects in Cohorts 2 and 3 will have skin biopsy samples taken from the calf at the following time-points: Cohort 2 (5 biopsies): Day 1 (within 3 hours pre-morning dose - duplicate biopsy), 12 hours after the last dose on Day 8, and at 168 hours (Day 15), and 504 hours (Day 29). Cohort 3 (5 biopsies): Day 1 (within 3 hours pre-morning dose), 12 hours after the last dose on Day 8, and at 168 hours (Day 15), 336 hours (Day 22) and 504 hours (Day 29). The samples taken from Cohort 2 will be used for intra-epidermal nerve fiber density assessments, whilst those taken from Cohort 3 will be used for PK assessments and metabolite ID. All biopsies are single samples unless otherwise specified.
^{l}All subjects in Cohort 3 will have urine collected within 3 hours before dosing (pre-morning dose) on Day 1 and from time 0 (predose) until 12 hours postdose on Day 8.
^{m}A 5-10 minute shower will be required between 10-11 hours after application of the morning dose on Days 1 to 8.
ⁿSubjects may be discharged from the clinic on Day 10 (approximately 48 hours after the last dose) upon safety review and completion of study requirements.

Subjects who meet the inclusion/exclusion criteria at Visit 1 will continue to Visit 2. Visit 2 consists of 11 days (Days −1 to Day 10) and 10 overnight stays, the procedures performed on each day are described as follows.
Day −1: The following procedures will be performed on Day −1:
   review inclusion/exclusion criteria;
   adverse event inquiry;
   concomitant medication review;
   perform symptom-based physical examination;
   perform 12-lead ECG;
   perform vital signs measurements;
   perform urine drug screen/alcohol/cotinine test; and
   perform serum pregnancy test (females only).
      A subject who does not meet study entry criteria will not be considered for screening again. Subjects who continue to meet the inclusion/exclusion criteria will be assigned a permanent unique randomization number and a treatment number.
Day 1: The following procedures will be performed prior to the first dose administration on Day 1:
   concomitant medication review;
   adverse event inquiry;
   perform vital signs measurements;
   collect blood sample for pharmacogenomics;
   collect blood sample for pharmacokinetics;
   collect blood sample for metabolite identification;
   collect urine sample for pharmacokinetics/metabolite identification (Group 3 only);
   Perform skin biopsies (Groups 2 and 3 only; Group 2 will have duplicate biopsies; Group 3 will have a single biopsy); and
   perform baseline dermal irritation assessment.
      Following completion of predose assessments, the study composition will be administered to the appropriate areas (morning dose) as defined for each Group in Table 7.
      The following procedures will be performed after the morning dose on Day 1:
   collect blood samples for pharmacokinetics;
   request subjects to take a shower 10 to 11 hours after the morning dose (i.e., 1 to 2 hours prior to the evening dose);
      An evening dose of the study composition will be administered on Day 1 12 hours after the morning dose.
Day 2: The following procedures will be performed prior to the morning dose administration on Day 2:
   concomitant medication review;
   adverse event inquiry;
   perform vital signs measurements;
   collect blood sample for pharmacokinetics; and
   perform dermal irritation assessment.
      Following completion of predose assessments, the study composition will be administered.
      The following procedures will be performed after the morning dose on Day 2:
   request subjects to take a shower 10 to 11 hours after the morning dose.
      An evening dose of the study composition will be administered 12 hours after the morning dose on Day 2.
Day 3: The following procedures will be performed prior to the morning dose administration on Day 3:
   concomitant medication review;
   adverse event inquiry;
   perform 12-lead ECG;
   perform clinical laboratory tests;
   perform vital signs measurements;
   collect blood sample for pharmacokinetics; and
   perform dermal irritation assessment.
      Following completion of predose assessments, the study composition will be administered.
      The following procedures will be performed after the morning dose on Day 3:
   request subjects take a shower 10 to 11 hours after the morning dose.
      An evening dose of the study composition will be administered 12 hours after the morning dose on Day 3.
Day 4: The following procedures will be performed prior to the morning dose administration on Day 4:
   concomitant medication review;
   adverse event inquiry;
   perform vital signs measurements;
   collect blood sample for pharmacokinetics; and
   perform dermal irritation assessment.
      Following completion of predose assessments, the study composition will be administered.
      The following procedures will be performed after the morning dose on Day 4:
   request subjects take a shower 10 to 11 hours after the morning dose.
      An evening dose of the study composition will be administered 12 hours after the morning dose on Day 4.
Day 5: The following procedures will be performed prior to the morning dose administration on Day 5:
   concomitant medication review;
   adverse event inquiry;
   perform serum pregnancy test (females only);
   perform vital signs measurements;
   collect blood sample for pharmacokinetics; and
   perform dermal irritation assessment.
      Following completion of predose assessments, the study composition will be administered.
      The following procedures will be performed after the morning dose on Day 5:
   request subjects take a shower 10 to 11 hours after the morning dose.
      An evening dose of the study composition will be administered 12 hours after the morning dose on Day 5.
Day 6: The following procedures will be performed prior to the morning dose administration on Day 6:
   concomitant medication review;
   adverse event inquiry;
   perform 12-lead ECG;
   perform vital signs measurements;
   collect blood sample for pharmacokinetics; and
   perform dermal irritation assessment.
      Following completion of predose assessments, the study composition will be administered.
      The following procedures will be performed after the morning dose on Day 6:
   request subjects take a shower 10 to 11 hours after the morning dose.
      An evening dose of the study composition will be administered 12 hours after the morning dose.
Day 7: The following procedures will be performed prior to the morning dose administration on Day 7:
   concomitant medication review;
   adverse event inquiry;
   perform vital signs measurements;
   collect blood sample for pharmacokinetics; and
   perform dermal irritation assessment.
      Following completion of predose assessments, the study composition will be administered.
      The following procedures will be performed after the morning dose on Day 7:
   request subjects take a shower 10 to 11 hours after the morning dose.
      An evening dose of the study composition will be administered 12 hours after the morning dose on Day 7.
Day 8: The following procedures will be performed prior to the final dose administration on Day 8:
   concomitant medication review;
   adverse event inquiry;
   perform 12-lead ECG;
   perform clinical laboratory tests;
   perform vital signs measurements;
   collect blood sample for pharmacokinetics; and
   perform dermal irritation assessment.
      Following completion of predose assessments, the study composition will be administered.
      The following procedures will be performed after the morning dose on Day 8: perform 12-lead ECGs;
   collect blood samples for pharmacokinetics;
   collect blood samples for metabolite identification;
   collect urine over 0 to 12 hours for pharmacokinetics/metabolite identification (Group 3 only);
   perform skin biopsy (Group 2 and 3 only);
   request subjects take a shower 10 to 11 hours after the morning dose on Day 8.
Day 9: The following procedures will be performed:
   concomitant medication review;
   adverse event inquiry;
   perform vital signs measurements;
   collect blood sample for pharmacokinetics;
   collect blood sample for metabolite identification; and
   perform dermal irritation assessment.
Day 10: The following procedures will be performed:
   concomitant medication review
   adverse event inquiry
   perform 12-lead ECG
   perform clinical laboratory tests
   perform vital signs measurements
   collect blood sample for pharmacokinetics
   collect blood sample for metabolite identification
   perform dermal irritation assessment
      After completion of these assessments, subjects will be reminded of all ongoing study restrictions/requirements and then discharged from the clinic.
Day 12 (Visit 3): The following procedures will be performed:
   concomitant medication review;
   adverse event inquiry;
   perform 12-lead ECG;
   perform vital signs measurements;
   collect blood sample for pharmacokinetics; and
   perform dermal irritation assessment.
Day 15 (Visit 4): The following procedures will be performed:
   concomitant medication review;
   adverse event inquiry;
   perform 12-lead ECG;
   perform vital signs measurements;
   collect blood sample for pharmacokinetics;
   perform dermal irritation assessment; and
   perform skin biopsy (Groups 2 and 3 only)
Day 22 (Visit 5): The following procedures will be performed:
   concomitant medication review;
   adverse event inquiry;
   perform 12-lead ECG;
   perform vital signs measurements;
   collect blood sample for pharmacokinetics;
   perform dermal irritation assessment; and
   perform skin biopsy (Group 3 only)
      Day 29 (Visit 6): The following procedures will be performed:
   concomitant medication review;
   adverse event inquiry;
   perform 12-lead ECG;
   perform vital signs measurements;
   collect blood sample for pharmacokinetics;
   perform dermal irritation assessment; and
   perform skin biopsy (Groups 2 and 3 only)
Day 39±2 (Follow-up Visit; Visit 7): The following procedures will be performed:
   concomitant medication review;
   adverse event inquiry;
   perform symptom-based physical examination;
   perform 12-lead ECG;
   perform clinical laboratory tests;
   perform vital signs measurements;
   perform serum pregnancy test (females only);
   collect blood sample for pharmacokinetics; and
   perform dermal irritation assessment.

Subjects with ongoing adverse events or clinically significant abnormal laboratory test results (as interpreted by the investigator) will be monitored accordingly, otherwise, the Follow-up visit will be the last study visit.

### Restrictions on Subjects During Study Period

Subjects will be required to comply with the following restrictions:

Subjects will be directed not to rub, or massage, or compress any of the application sites after study composition administration. Subjects will be instructed to try not to touch the application sites until the composition has been absorbed into the skin. Subjects will be required to cover an application site with socks and light, loose-fitting clothing such as a tee shirt and sweat pants, beginning 30 minutes after each application. Likewise, subjects will be allowed to lie on an application site after covering the site with light clothing beginning 30 minutes after each application.

Subjects may not shave or wax the application sites for the duration of the study up until discharge at the end of the inpatient phase. Shaving and waxing is allowed during the outpatient phase. Subjects may not undergo laser hair removal of the application sites for the duration of the study including follow-up.

Subjects will not be allowed to undergo any vigorous physical activities during the inpatient phase of the study, especially activities that would promote sweating (e.g., running, weight-lifting). Likewise, swimming and use of hot-tubs, steam baths, saunas, and tanning beds is prohibited during the inpatient phase of the study.

Bathing is prohibited during the inpatient phase of the study. However, during the inpatient phase, subjects will be allowed to shower for 5 to 10 minutes each day, in the evening 10 to 11 hours after the morning dose of study composition on Days 1 to 8 (i.e., 1 to 2 hours prior to evening dose where applicable). Subjects may use soap and shampoo whilst showering. Beginning on Day 9, subjects may shower for 5 to 10 minutes either in the morning or evening at their discretion. In addition, upon discharge from the clinic, subjects may resume normal bathing activities without restriction.

Standard meals will be supplied throughout the inpatient phase of the treatment period. Subjects may not consume alcohol, grapefruit juice, or caffeine throughout the inpatient phase of the study. Excessive consumption of coffee, tea, and/or caffeine-containing beverages or food (i.e., 600 mg of caffeine or more per day, or 5 or more cups of coffee per day) will be prohibited for a minimum of 2 weeks before the first dose of study composition and throughout the study, including follow-up.

Subjects may not smoke or use any tobacco/nicotine products for the duration of the study, including follow-up.

### Assessment of Pharmacokinetics

The following pharmacokinetic variables will be calculated for the spiro-oxindole compound using appropriate non-compartmental methods, if the data permit. Actual sampling times will be used in the analysis:
Cₘₐₓ maximum plasma concentration (Days 1 and 8)
Tₘₐₓ time-of-maximum concentration (Days 1 and 8)
AUC_{τ} area under the concentration time profile over the dosing interval (Days 1 and 8)
Cₘᵢₙ morning predose concentrations (Days 1 to 8)
Cₐᵥₑ average plasma concentration over the dosing interval (Days 1 and 8)
t_{1/2} apparent terminal half-life after the last dose.
λ_{z} terminal elimination rate constant after the last dose
R_{ac, AUCτ} accumulation ratio: AUC_{τ(Day 8)}/AUC_{τ(Day 1)}
R_{ac, Cmax} accumulation ratio: C_{max(Day 8)}/C_{max(Day 1)}
   Additional parameters may also be calculated.

The amount of the spiro-oxindole compound in urine will be expressed in terms of the amount collected over 12 hours.

Concentrations of the spiro-oxindole compound in the skin biopsies from Group 3 will be determined and, if possible, the half-life of the spiro-oxindole compound from skin will be estimated.

Plasma, urine and skin samples will also be used for exploratory metabolite identification if possible. These data will be reported independently and will not be included in the clinical study report.

### Samples and Handling

All subjects will have blood samples (approximately 3 mL) collected via venipuncture or indwelling catheter for plasma concentrations of the spiro-oxindole compound at the following times:
Day 1: 0 (pre-morning dose), 1, 2, 3, 4, 6, 8, 10 and 12 hours post-morning dose
Days 2, 3, 4, 5, 6 and 7: 0 (pre-morning dose)
Day 8 to 29: 0 (pre-final dose), and at the following times after the final dose: 1, 2, 3, 4, 6, 8, 10, 12, 16, 24, 30, 36, 48, 96 (Day 12), 168 (Day 15), 336 (Day 22), 504 (Day 29), and 744±48 (Day 39±2) hours.

All subjects will have an additional blood sample (2 mL) collected for exploratory metabolite identification at the following times:
Day 1: 0 (pre-morning dose)
Day 8: 1, 2, 6, 12, 24 and 48 hours after the final dose.

The dates and times of study drug administration and the date and time of each pharmacokinetic sample will be recorded on the source documentation.

Permissible windows for pharmacokinetic samples are as follows:

within 15 minutes prior to dosing for predose samples
±1 minute for samples taken from predose to 4 hours postdose
±2 minutes for samples taken from 4 hours to 10 hours postdose
±5 minutes for samples taken from >10 hours to 24 hours postdose
±10 minutes for samples taken from >24 hours to 48 hours postdose
±4 hours for all outpatient visits on Days 12, 15, 22 and 29±
±48 hours for Day 39.

Samples will be collected into K₂EDTA VACUTAINER® (Becton, Dickinson and Company) tubes, inverted slowly 6 to 8 times to mix the contents, and placed on ice (0° C. to 5° C.). Blood samples will be centrifuged (1500 g, approximately 10 minutes, 0° C. to 5° C.) within 1 hour after sampling. Separated plasma from the pharmacokinetic samples will be transferred into 2 opaque, labeled, 2 mL polypropylene tubes (Sets A and B). A minimum of 500 μL plasma will be transferred to Set A and the remaining plasma (minimum of 500 μL) will be transferred to Set B. Separated plasma from the exploratory metabolite identification samples will be transferred into opaque, labeled, polypropylene tubes (set C).

Sample labels should include the study number, subject identification number, date, Day, nominal collection time, set (A, B or C), and indication that they are pharmacokinetic or metabolite identification plasma samples. Plasma samples will be placed on ice (0° C. to 5° C.) in an upright position until they are frozen at approximately −70° C. within 1 hour of centrifugation for later analysis.

Subjects in Group 3 will have urine samples collected at the following times:
Day 1: 0 (within 3 hours pre-morning dose)
Day 8: 0-12 hours postdose.

Two 10-mL aliquots (Set A and B) of the Day 1 sample will be transferred to labeled cryogenic storage tubes and frozen at approximately −70° C. within 1 hour of collection. On Day 8, subjects will be asked to void their bladder prior to the start of the collection period (i.e., within 15 minutes prior to dosing). All subsequent urine voids up to 12 hours postdose will be collected. The urine should be kept refrigerated at approximately 2 to 8° C. until the end of the collection period. At the end of the collection period, subjects will be asked to empty their bladder and the total volume of the complete urine collection will be recorded. The cumulative urine collection should be thoroughly mixed and two 10 mL aliquots (Sets A and B) will be transferred to labeled cryogenic storage tubes and frozen at approximately −70° C. within 1 hour of completion of the collection for later analysis. Sample labels should include the study number, subject identification number, date, Day, nominal collection time and indication that they are urine samples.

All subjects in Group 3 will have skin biopsy samples taken from the calf at the following timepoints for the purpose of pharmacokinetic analysis and metabolite identification if possible (5 biopsies): Day 1 (within 3 hours pre-morning dose), at 12 hours after the last dose on Day 8, and at 168 hours (Day 15), 336 hours (Day 22), and 504 hours (Day 29). A single sample will be taken at each timepoint. The allowed deviations from the nominal sampling times will be ±10 minutes on Day 8 and ±4 hours for outpatient visits (Days 15, 22 and 29).

Prior to each skin biopsy, the area to be biopsied will be washed with water to remove as much of the study composition as possible and then wiped gently dry. The cleansed area will then be wiped twice with an alcohol swab to remove any residual study composition and then allowed to dry. A local anesthetic (lidocaine) will be applied and a punch biopsy (2-4 mm skin each) will be obtained using the clinical site's best practices.

Each skin sample will be collected into storage tubes labelled with the study number, subject identification number, date, Day, nominal collection time and indication that they are skin samples for pharmacokinetics, and stored at approximately −70° C. until analyzed.

### Clinical Laboratory Tests

Clinical laboratory tests (serum chemistry and hematology) and urinalysis will be performed at the time points indicated in Table 8. Specific laboratory tests to be performed are listed below.

The following serum chemistry tests will be performed:

calcium

phosphorus

sodium

potassium

chloride

bicarbonate or carbon dioxide

glucose

blood urea nitrogen (BUN)

creatinine

cholesterol

uric acid

ALT

AST

lactic dehydrogenase (LDH)

gamma-glutamyl transpeptidase (GGT)

alkaline phosphatase

creatine phosphokinase

total protein

albumin

total bilirubin

direct bilirubin

indirect bilirubin

The following hematology tests will be performed:

hemoglobin

hematocrit

red blood cell (RBC) count

platelet count

absolute neutrophil count (ANC)

white blood cell (WBC) count and differential count

polymorphonuclear leukocytes (neutrophils)

lymphocytes

eosinophils

monocytes

basophils

atypical lymphocytes

Urinalysis will include testing for the following:

protein

glucose

ketones

blood (hemoglobin)

pH

specific gravity

microscopic

bacteria

RBCs

WBCs

casts

crystals

### Intra-Epidermal Nerve Fiber Density Samples

All subjects in Group 2 will have skin biopsy samples taken from the calf at the following timepoints for the purpose of intra-epidermal nerve fiber density assessment (5 biopsies): Day 1 (within 3 hours pre-morning dose), at 12 hours after the last dose on Day 8, at 168 hours (Day 15), and 504 hours (Day 29). On Day 1, duplicate biopsies will be taken to allow assessment of baseline variability. All other biopsies will be single samples.

The allowed deviations from the nominal sampling times will be ±10 minutes for Day 8 and ±4 hours for outpatient visits (Days 15 and 29).

Prior to each skin biopsy, the area to be biopsied will be washed with water to remove as much of the study composition as possible and then wiped gently dry. The cleansed area will then be wiped twice with an alcohol swab to remove any residual study composition and then allowed to dry. A local anesthetic (lidocaine) will be applied and a punch biopsy (2-4 mm skin each) will be obtained according to the clinical sites best practices.

Each skin sample for intra-epidermal nerve fiber density assessment will be placed in a vial pre-filled with fixative as per instructions of the clinical histology laboratory. The vial should be labelled with the study number, subject identification number, date, Day, nominal collection time and indication that they are skin samples for histological evaluation. These samples will not be frozen, but should be stored at approximately 2 to 8° C. until analyzed.

### Assessment of Pharmacogenomics

It is recognized that genomic variation within the population can be an important contributory factor to inter-individual differences in drug response. Pharmacogenomic (PGx) studies investigate the association between genetic polymorphisms and clinical response to a certain therapeutic intervention. It may help explain inter-individual variability and subsequently identify population subgroups that respond differently to the drug. Furthermore, regulatory guidance and white papers indicate that PGx analyses employing DNA collected from all study participants may support investigation of unexpected adverse events.

Samples for PGx will be obtained from all subjects in the study. PGx assessment will include assessment of CYP2C19 status of subjects and will potentially include association analysis of other DNA variations with clinical treatment responses to the spiro-oxindole compound (e.g., pharmacokinetics, tolerability, and safety features). The final list of genes that might be investigated will be selected in a later stage before the analysis so as to allow updating with new scientific information. Genomic analysis could also include sequencing of the whole genome if required.

PGx assessment will be performed using blood samples collected after randomization on Day 1.

### Statistical Considerations

For this study, no prospective calculations of statistical power have been made. A sample size of 45 healthy male and female subjects (15 per group) is chosen based on clinical and practical considerations. The sample size is intended to provide reasonably precise estimates of the systemic exposure of the spiro-oxindole compound following topical multiple-dose administration. No formal statistical analyses will be conducted. All data will be listed and summarized descriptively by cohort (dose) and presented in tabular and graphical form, where applicable.

### Expected Results

The results of this study are expected to show that concentrations of the spiro-oxindole in the skin tissues in the area where the 8% test pharmaceutical composition of the invention was administered to the subject are significantly higher than concentrations of the spiro-oxindole compound in the plasma of the subject following administration, thereby confirming that administration of a pharmaceutical composition of the invention results in minimal or negligible systemic exposure.

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

1. A method of treating pain associated with osteoarthritis of a joint in a mammal, wherein the method comprises administering to the affected joint of the mammal a topical pharmaceutical composition comprising one or more excipients and a therapeutically effective amount of a spiro-oxindole compound having the following formula:

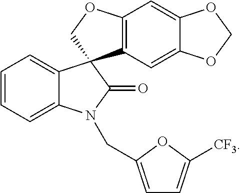 [SMILES: O=C1N(Cc2ccc(C(F)(F)F)o2)c2ccccc2[C@]12COc1cc3c(cc12)OCO3]

2. The method of claim 1 wherein the administration is periodic administration.

3. The method of claim 1 which results in minimal or negligible systemic exposure of the spiro-oxindole compound.

4. The method of claim 1 which results in a greater concentration of the spiro-oxindole compound in the synovial membrane and synovial fluid of the affected joint than the concentration of the spiro-oxindole compound in the plasma of the mammal.

5. The method of claim 1 wherein the osteoarthritis is primary osteoarthritis.

6. The method of claim 1 wherein the osteoarthritis is secondary osteoarthritis.

7. The method of claim 1, wherein the topical pharmaceutical composition comprises 2% to 8% (w/w) of the spiro-oxindole compound.

8. The method of claim 1 wherein the topical pharmaceutical composition comprises 45% to 55% (w/w) PEG 400; 5% to 15% (w/w) Transcutol® P; 2.5% to 7.5% (w/w) oleyl alcohol; 2.5% to 7.5% (w/w) isopropyl myristate; 0.1% w/w to 7.5% (w/w) stearyl alcohol; 0.05% to 1% (w/w) butylated hydroxytoluene; and 15% to 30% (w/w) PEG 3350.

9. The method of claim 1, wherein the topical pharmaceutical composition comprises 2.0% (w/w) of the spiro-oxindole compound; 52.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350.

10. The method of claim 1, wherein the topical pharmaceutical composition comprises 4.0% (w/w) of the spiro-oxindole compound; 50.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350.

11. The method of claim 1, wherein the topical pharmaceutical composition comprises 8.0% (w/w) of the spiro-oxindole compound; 46.9% (w/w) PEG 400; 10% (w/w) Transcutol® P; 5% (w/w) oleyl alcohol; 5% (w/w) isopropyl myristate; 5% (w/w) stearyl alcohol; 0.1% (w/w) butylated hydroxytoluene; and 20% (w/w) PEG 3350.

12. The method of claim 2 wherein the periodic administration is once a day, twice a day, three times a day or four times a day.

13. The method of claim 2 wherein the periodic administration is once a day.

14. The method of claim 2 wherein the periodic administration is twice a day.

15. The method of claim 1 wherein the topical pharmaceutical composition is administered to the skin over and surrounding the affected joint in a dose volume of from about 1.0 μL/cm² to about 9.0 μL/cm².

16. The method of claim 15 wherein the dose volume is from about 1.0 μL/cm² to about 4.0 μL/cm² of skin.

17. The method of claim 16 wherein the dose volume is 3.0 μL/cm².

18. The method of claim 2 wherein the therapeutically effective amount of the topical pharmaceutical composition is from about 500 mg to about 2000 mg per each periodic administration to the affected joint.

19. The method of claim 18 wherein the therapeutically effective amount of the topical pharmaceutical composition is about 1200 mg per each periodic administration to the affected joint.

20. The method of claim 1 wherein the therapeutically effective amount of the topical pharmaceutical composition is effective in reducing the severity of the pain or alleviating the pain.

21. The method of claim 1 wherein the affected joint is a knee.

22. The method of claim 1 wherein the affected joint is a joint in a hand.

23. The method of claim 1 wherein the affected joint is an ankle.

24. The method of claim 1 wherein the affected joint is a joint in the shoulder.

25. The method of claim 1 wherein the affected joint is a hip.

26. The method of claim 2 wherein the mammal is human.

27. The method of claim 26 wherein the periodic administration of the topical pharmaceutical composition is effective in reducing an average evening pain intensity or severity in the affected joint in the human when walking on a flat surface when compared to baseline pain intensity.

28. The method of claim 26 wherein the periodic administration of the topical pharmaceutical composition is effective in reducing an average daily pain intensity or severity in the affected joint in the human when walking on a flat surface when compared to baseline pain intensity.

29. The method of claim 26 wherein the periodic administration of the topical pharmaceutical composition is effective in reducing an average morning pain intensity or severity in the affected joint in the human when walking on a flat surface when compared to baseline pain intensity.

30. The method of claim 26 wherein the periodic administration of the topical pharmaceutical composition is effective in increasing physical function of the affected joint during a daily activity when compared to baseline physical function.

31. The method of claim 26 wherein the periodic administration of the topical pharmaceutical composition is effective in reducing stiffness of the affected joint when compared to baseline stiffness.

32. The method of claim 26 wherein the periodic administration of the topical pharmaceutical composition is effective in reducing the intensity or severity of the pain in the affected joint as assessed by a WOMAC, PQAS-R or OMERACT-OARSI questionnaire.

33. The method of claim 32 wherein the periodic administration of the topical composition is effective in reducing the intensity or severity of the pain by 30% when compared to the baseline intensity or severity of the pain.

34. The method of claim 32 wherein the periodic administration of the topical composition is effective in reducing the intensity or severity of the pain by 50% when compared to the baseline intensity or severity of the pain.

35. A method of locally treating pain associated with osteoarthritis of a joint in a mammal with a minimal or negligible systemic exposure, wherein the method comprises increasing the concentration of a spiro-oxindole compound having the following formula: 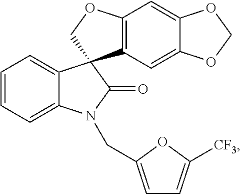 [SMILES: O=C1N(Cc2ccc(C(F)(F)F)o2)c2ccccc2[C@]12COc1cc3c(cc12)OCO3]
to a therapeutically effective amount in the synovial membrane of the affected joint in the mammal by administering to the affected joint a topical pharmaceutical composition comprising one or more excipients and a therapeutically effective amount of the spiro-oxindole compound.

36. The method of claim 35 where the administration is periodic administration.

37. The method of claim 36 wherein the joint is a knee, a joint in a hand, an ankle, a joint in the shoulder or a hip.

38. The method of claim 35 wherein the mammal is a human.

39. A method of treating pain associated with osteoarthritis of a joint in a mammal, wherein the method comprises administering to the affected joint of the mammal a topical pharmaceutical composition comprising one or more excipients and a therapeutically effective amount of a spiro-oxindole compound having the following formula: 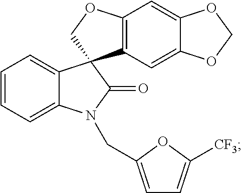 [SMILES: O=C1N(Cc2ccc(C(F)(F)F)o2)c2ccccc2[C@]12COc1cc3c(cc12)OCO3]
and a therapeutically effective amount of one or more other therapeutic agents.

40. The method of claim 39 where the administration is periodic administration.

41. The method of claim 39 wherein the one or more other therapeutic agent is acetaminophen.
